(19) United States
(12) Patent Application Publication
(10) Pub. No.: US 2024/0148910 A1
(43) Pub. Date: May 9, 2024

(54) HYDROPORPHYRIN-DOPED NEAR-INFRARED-EMITTING POLYMER DOTS FOR CELLULAR FLUORESCENCE IMAGING

(71) Applicant: UNIVERSITY OF MARYLAND, BALTIMORE COUNTY, Baltimore, MD (US)

(72) Inventors: Zeev Rosenzweig, Baltimore, MD (US); Marcin Ptaszek, Baltimore, MD (US); Connor Riahin, Baltimore, MD (US); Adam Meares, Baltimore, MD (US)

(21) Appl. No.: 18/495,141

(22) Filed: Oct. 26, 2023

Related U.S. Application Data
(60) Provisional application No. 63/380,996, filed on Nov. 7, 2022.

(51) Int. Cl. A61K 49/00

(52) U.S. Cl. CPC A61K 49/0045; A61K 49/0067

(57) ABSTRACT

The present invention relates to nanoscale polymer dots (Pdots) that include strongly light absorbing semiconducting polymer nanoparticles doped with near-IR emitting dyes. The polymer functions as an antenna and transfers the excitation energy to the doped near-IR emitting dye molecules, which function as the emitting entity. The resulting Pdots feature very bright near-IR emission with emission wavelength tunability, high water solubility, and stability in biological solutions.

### CROSS REFERENCE TO RELATED APPLICATIONS

This application claims priority to U.S. Provisional Patent Application No. 63/380,996 filed on Nov. 7, 2022 in the name of Zeev ROSENZWEIG et al. entitled “HYDROPORPHYRIN-DOPED NEAR-INFRARED-EMITTING POLYMER DOTS FOR CELLULAR FLUORESCENCE IMAGING,” which is hereby incorporated by reference herein in its entirety.

### STATEMENT OF FEDERALLY SPONSORED RESEARCH

This invention was made with government support under Grant No. UNIV0045 awarded by the Department of Energy. The government has certain rights in the invention.

### FIELD

The present invention relates to nanoscale polymer dots (Pdots) that include strongly light absorbing semiconducting polymer nanoparticles doped with near-IR emitting dyes.

### BACKGROUND

Fluorescence bioimaging methods, which often rely on the availability of fluorescent probes, are noninvasive, do not require harmful radiation, and offer high spatial resolution and temporal resolution when used to visualize cells, tissues, and even whole animals. Near-infrared (NIR) fluorescent probes are particularly desirable due to lower levels of light scattering with increasing excitation wavelength, reduced tissue autofluorescence in the NIR region of the electromagnetic spectrum, and increased penetration depth and a signal-to-noise ratio, which enables quantitative fluorescence bioimaging of cells and tissues.

Several classes of NIR dyes have been examined for in vivo imaging, including small organic molecules used as NIR fluorophores including cyanines and related dyes, BODIPY and aza-BODIPY, squaraines, porphyrin derivatives, and benzobisb[1,2,5]thiadiazole. Organic dyes have certain advantages, but they suffer from significant drawbacks, which include poor water solubility, low emission quantum yield when modified to render them water soluble, low photostability, and limited tunability of optical properties. In addition, the optical properties of currently available fluorophores limit their applications for in vivo multicolor imaging, since many NIR agents feature broad emission bands and relatively narrow absorption bands. There is a growing need for new bright, photostable, tunable NIR fluorophores, with optical properties suitable for bioimaging applications in biological aqueous media.

NIR-emitting nanoparticles, for example, luminescent semiconductor quantum dots (QDs), were developed as an alternative to NIR-emitting molecular probes to overcome their limitations. A major limitation of QDs, however, is their inherent toxicity, primarily due to (1) leaching of toxic metal ions from the QD core and (2) adverse interactions with the ligands used to stabilize and impart aqueous solubility to the QDs. As such, attention within the field has shifted to the development of new nontoxic fluorescent nanoparticles. Silica nanoparticles do not have an obvious biological degradation pathway, which precludes their use in vivo. Carbon dots are a nontoxic alternative to inorganic Pdots. However, it is difficult to tune their emission color, which requires an elaborate separation protocol following synthesis.

Another disadvantage of traditional NIR fluorescent dyes is they generally suffer from poor water solubility, and structural modifications to improve their water solubility tend to significantly decrease their emission quantum yield. Recently, semiconducting polymer nanoparticles, named polymer dots (Pdots), have generated increased interest as a new type of fluorophore, which could provide a viable alternative and overcome some of the major drawbacks of molecular fluorophores and luminescent QDs in the visible and NIR regions. Pdots are composed of semiconducting polymers that offer several advantages over inorganic QDs and organic dyes, including facile synthesis, biocompatibility, less batch-to-batch variance in emission properties, high photostability, tunable absorption and emission, and exceptionally high absorptivity and brightness [J. Pecher, et al., 2010]. On a per-particle basis, fluorescent Pdots are several times brighter than similar emitting QDs and organic dyes. This brightness is the result of fast emissive rates, large extinction coefficients, and high emission quantum yields. Additionally, Pdots are resistant to photobleaching, making them highly applicable to fluorescence imaging and sensing applications. Finally, Pdots exhibit minimal or no cytotoxicity towards mammalian cells which distinguish them from heavy metal, cadmium and lead containing quantum dots.

Pdots can be synthesized using a nanoprecipitation method by adding highly emitting hydrophobic polymer molecules dissolved in an organic solvent like tetrahydrofuran (THF) to aqueous solution under vigorous sonication [T. Xu, et al., 2014]. Initial studies using this approach resulted in Pdots characterized by broad emission and low emission quantum yield [C. V. Rohatgi, et al., 2018]. Modifications of the polymer molecular structure such as the inclusion of π-bridges [C.-S. Ke, et al., 2017] and donor-donor-acceptor systems [L. Chen, et al., 2019] have recently improved the emission quantum yield of Pdots made using this approach. Another approach involves either doping or covalently attaching a NIR-emitting dye to the Pdots [Y. Jin, et al., 2011; D. Chen, et al., 2017; S. Chen, et al., 2018; N. Gupta, et al., 2021; N. R. Paisley, et al., 2021]. Doping alters the emission properties of the Pdots due to energy transfer between the polymer and the doped dye molecules. The polymer molecules absorb the excitation light and transfer the energy to the dye molecules. This approach takes full advantage of the properties of both materials: the strong, broad absorption of the polymer and the narrow NIR emission of the dye. Pdots synthesized with this method are significantly brighter on a per-particle basis than the dye alone or similar emitting QDs. Disadvantageously, a key limitation of NIR-emitting Pdots is the limited availability of NIR dyes and, consequently, the truncated range of possible emission peak wavelengths of NIR-emitting Pdots.

Therefore, there continues to be a need for NIR-emitting dyes that could be used as efficient acceptors in Pdots, which will help realize the multiplexing potential of NIR-emitting Pdots. A series of hydroporphyrin or benzo-bis-thiadiazole (BBTD)-doped Pdots, which can be excited by a single excitation source, is described herein. Systematic modifications of the doping dyes result in emission wavelength tunability between 640 and 1000 nm. The utility of these NIR-emitting Pdots includes, but is not limited to, bioimaging probes in cellular imaging applications and as super-resolution imaging NIR probes.

### SUMMARY

In some aspects, the present invention relates to a method of making near infrared (NIR)-emitting polymer dots (Pdots), said method comprising:
dissolving at least one semiconducting polymer, optionally at least one additional component, and at least one doping dye in at least one water-miscible organic solvent to form a reaction mixture; and
coprecipitating the at least one semiconducting polymer, optionally at least one additional component, and at least one doping dye to form NIR-emitting Pdots by injecting the reaction mixture into a volume of water under sonication. In some embodiments, the at least one doping dye comprises a hydroporphyrin dye having varying auxochromes or a benzo-bis-thiadiazole (BBTD) dye having varying functional groups.

In other aspects, the present invention relates to near infrared (NIR)-emitting polymer dots (Pdots) comprising at least one semiconducting polymer, optionally at least one additional component, and at least one doping dye, wherein the at least one doping dye comprises a hydroporphyrin dye having varying auxochromes or a benzo-bis-thiadiazole (BBTD) dye having varying functional groups.

In still another aspect, the present invention relates to antibody-Pdots conjugate comprising at least one NIR-emitting Pdot and at least one antibody, wherein the at least one NIR-emitting Pdot comprises at least one semiconducting polymer, optionally at least one additional component, and at least one doping dye, wherein the at least one doping dye comprises a hydroporphyrin dye having varying auxochromes or a benzo-bis-thiadiazole (BBTD) dye having varying functional groups.

In yet another aspect, the present invention relates to a method of making an antibody-Pdots conjugate, said method comprising:
mixing an amount of a solution comprising NIR-emitting Pdots with a poly(ethylene) glycol (PEG) solution and a buffer to form a first mixture;
adding a solution comprising an antibody to the first mixture with mixing to form a second solution; and
adding an amount of N-(3-Dimethylaminopropyl)-NO-ethylcarbodiimide (EDC) with agitation to effectuate a coupling reaction to conjugate the NIR-emitting Pdots with the antibody. In some embodiments, the at least one NIR-emitting Pdots comprise at least one semiconducting polymer, optionally at least one additional component, and at least one doping dye, wherein the at least one doping dye comprises a hydroporphyrin dye having varying auxochromes or a benzo-bis-thiadiazole (BBTD) dye having varying functional groups.

In still another aspect, the present invention relates to a method of using NIR-emitting Pdots for enhanced fluorescence imaging of one or more targets, said method comprising incubating a mixture comprising one or more NIR-emitting Pdots for a time to effectuate incorporation of the one or more NIR-emitting Pdots into and/or onto the target, and imaging said target using fluorescence measurement means.

Other aspects, features and embodiments of the invention will be more fully apparent from the ensuing disclosure and appended claims.

### BRIEF DESCRIPTION OF THE FIGURES

FIG. 1. Chemical structures of hydroporphyrins (phthalocyanines, chlorins and bacteriochlorins) used to prepare the dye-doped Pdots. The notation PXXX stands for porphyrin and its peak emission wavelength.

FIG. 2A. Absorption (black) and fluorescence (red) spectra of PFBT.

FIG. 2B. Absorption (black) and fluorescence (red) spectra of ADS104.

FIG. 2C. Absorption (black) and fluorescence (red) spectra of ADS 111.

FIG. 2D. Absorption (black) and fluorescence (red) spectra of ADS113.

FIG. 3. Preparation of dye-doped NIR-emitting polymer dots (Pdots), shown here with P720.

FIG. 4A. Size and morphological characterization of PFBT polymer dots (loaded with P640) by TEM. The representative TEM image shows the near-spherical Pdots to average 46±12 nm in diameter with some disorder.

FIG. 4B. Histogram of Pdots' size distribution from TEM images (n=69) shows the Pdots to be somewhat polydispersed and the formation of larger aggregates.

FIG. 4C. Dynamic light scattering (DLS) measurements show the Pdots' hydrodynamic diameter to an average of 52 nm, which is in close agreement with the TEM results.

FIG. 5. Normalized emission spectra of porphyrin-doped polymer dots (termed PPDXXX) (λₑₓ=450 nm).

FIG. 6A. Live/dead rat endothelial cells (RECs) incubated for 1 h at 37° C. in the presence of P640-loaded Pdots at 0 μg/mL (scale bar: 200 μm). Calcein-AM (green) stain is used to stain live cells and ethidium homodimer-1 (red) is used to stain dead cells.

FIG. 6B. Live/dead RECs incubated for 1 h at 37° C. in the presence of P640-loaded Pdots at 10 g/mL (scale bar: 200 μm).

FIG. 6C. FIG. 6A. Live/dead RECs incubated for 1 h at 37° C. in the presence of P640-loaded Pdots at 50 μg/mL (scale bar: 200 μm).

FIG. 6D. Survival percentage of RECs with Pdots at different concentrations.

FIG. 7A. Bright-field of RECs incubated with one type of NIR-emitting Pdots PPD640.

FIG. 7B. Fluorescence images of rat endothelial cells incubated with one type of NIR-emitting Pdots PPD640. Inset shows the fluorescence spectra of subsections of the image with high Pdot density.

FIG. 7C. Bright-field of rat endothelial cells incubated with two types of NIR-emitting Pdots PPD640 and PPD660.

FIG. 7D. Fluorescence images of rat endothelial cells incubated with two types of NIR-emitting Pdots PPD640 and PPD660. Inset shows the fluorescence spectra of subsections of the image with high Pdot density.

FIG. 7E. Bright-field of rat endothelial cells incubated with three types of NIR-emitting Pdots PPD640, PPD660, and PPD690.

FIG. 7F. Fluorescence images of rat endothelial cells incubated with three types of NIR-emitting Pdots PPD640, PPD660, and PPD690. Inset shows the fluorescence spectra of subsections of the image with high Pdot density.

FIG. 8A. A schematic of Flagellin Sensitive2 (FLS2) expressing plant root hair cells labeled with antibody-conjugated NIR emitting polymer dots.

FIG. 8B. Example of fluorescence images showing leaf cells taken from A. thaliana expressing FLS2 receptor kinase tagged with FLAG at the N-terminal (left panel) and from a wild-type plant expressing FLS2 with no tag serving as the negative control (right panel). The receptor is labeled using a primary antibody against FLAG, followed by PPD690 (Pdots encapsulating the P690 dye), and decorated with a secondary antibody against the primary antibody. Images were taken in two channels to capture the emissions of the polymer (647±57 nm, upper) and the emission of the dye (710±40 nm, lower). Scale bars represent 20 μm.

FIG. 8C. Example of fluorescence images showing root hair cells taken from A. thaliana expressing Flagellin Sensitive2 (FLS2) receptor kinase tagged with FLAG at the N-terminal (left panel) and from a wild-type plant expressing FLS2 with no tag serving as the negative control (right panel). Materials and methods are otherwise the same as those recited in FIG. 8B.

FIG. 9. Demonstration of Pdot blinking. Consecutive images of PPD640 Pdots were collected from a series shown in a video. The Pdots were diluted and imaged on a glass coverslip using a 440 nm excitation wavelength and an ˜600 to 700 nm emission band. The arrows point to two Pdots as an example of their blinking through the series.

FIG. 10. Molecular structures of BBTD dyes used to form Pdots for NIR fluorescence imaging between 780 and 1000 nm.

FIG. 11A. Representative Cryo-TEM images of unmodified porphyrin-doped Pdots.

FIG. 11B. Representative Cryo-TEM images of antibody-functionalized porphyrin-doped Pdots. The circle indicates a confirmed antibody, while the arrows indicate possible antibodies on the Pdots' surface.

FIG. 12A. Absorption spectra of BBTD-Pdots. The spectra are dominated by the absorption of PFBT at 450 nm but lower absorption peaks of BBTD dyes at longer wavelengths between 600 and 700 nm are also seen.

FIG. 12B. Fluorescence spectra of BBTD-Pdots (λex=450 nm), demonstrating near infrared emission with emission wavelengths between 800 and 1000 nm.

FIG. 13A. Molecular structures of the blue-emitting polymer and Ru(dpp)₃²⁺ used to form oxygen sensitive Pdots.

FIG. 13B. Fluorescence spectra of oxygen sensing Ru(dpp)₃²⁺-Pdots demonstrating their oxygen sensitivity.

FIG. 13C. Demonstration of the high reversibility of Ru(dpp)₃²⁺-Pdots when subjected to five cycles of saturating Ru(dpp)₃²⁺-Pdots aqueous solution with nitrogen and oxygen.

FIG. 14A. Evaluation of fluorescence intensity of polymer dots (Pdots) by different excitation filters in vitro. Graphs of the emission spectra of four Pdots (P660, P690, P700 and P720) using different excitation (ex) band-pass filters (blue ex filter, 445-490 nm; blue-green ex filter, 480-520 nm; green ex filter, 503-555 nm; green-yellow ex filter, 540-580 nm).

FIG. 14B. Peak emission intensity of each Pdot with different excited conditions (n=3, mean±standard deviation). ex: Excitation.

FIG. 15A. Graph of the emission spectra for each of the four polymer dots (Pdots) used.

FIG. 15B. In vitro spectral fluorescence imaging with four Pdots. Spectrally resolved images are shown for each Pdot. The composite image of P660 (cyan), P690 (green), P700 (yellow) and P720 (magenta) is shown at the lower left. Scale bar=1 cm.

FIG. 16A. In vivo spectral fluorescence drainage imaging of a mouse injected intracutaneously with four polymer dots (Pdots; 660, cyan; 690, green; 700, yellow; 710, magenta) into the middle phalanges of the bilateral upper extremities and each ear (shown in the schema). White image, spectral-resolved images for each Pdot, composite image of Pdots, merged image of white and composite image are shown. Scale bar=1 cm.

FIG. 16B. Ex vivo spectral fluorescence imaging of the four draining lymph nodes after surgical resection. White image, spectral-resolved images for each Pdot and composite image of Pdots are shown. Scale bar=1 cm.

### DETAILED DESCRIPTION, AND PREFERRED EMBODIMENTS THEREOF

Although the claimed subject matter will be described in terms of certain embodiments, other embodiments, including embodiments that do not provide all of the benefits and features set forth herein, are within the scope of this disclosure as well. Various structural and parameter changes may be made without departing from the scope of this disclosure.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Subject” as used herein refers to any vertebrate such as mammals, birds, reptiles, amphibians and fish including, but not limited to, a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, monkey, chimpanzee, and humans. In some embodiments, the subject is a human.

As defined herein, a “semiconductor polymer dot,” or a “Pdot” is a nanoparticle having a diameter from about 10 nm to 100 nm, and they comprise semiconducting polymer and at least one additional additive to enable their aqueous solubility and unique emission properties.

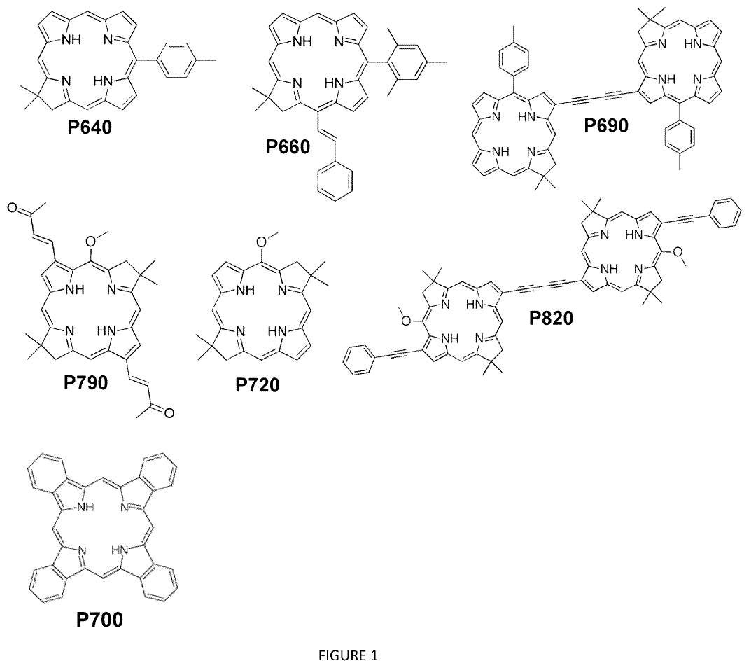

For the purposes of the present description, “hydroporphyrins” (e.g., chlorins and bacteriochlorins, FIG. 1) have attractive optical properties for multiplexed cellular imaging applications. Chlorins and bacteriochlorins are synthetic analogues of the photosynthetic pigments chlorophylls and bacteriochlorophylls. These tetrapyrrolic macrocycles differ from well-known porphyrins by having one (“chlorins”) or two (“bacteriochlorins”) partially saturated pyrroline rings. They are characterized by relatively strong absorption in the deep red (630-690 nm, ε˜40 000-80 000 M⁻¹·cm⁻¹ for chlorins) or NIR (700-800 nm, ε˜100 000-120 000 M⁻¹·cm⁻¹ for bacteriochlorins). The emission spectra of both are relatively narrow (FWHM ˜12-25 nm) with quantum yield Φ_{f}˜0.20-0.40 (chlorins) and 0.10-0.25 (bacteriochlorins). The absorption and emission wavelengths can be tuned across a broad spectral range by either (a) substitution at 3,13-pyrrolic positions of the macrocyclic ring with conjugated, electron-withdrawing, or electron-donating substituents [E. Yang, et al., 2011; H. L. Kee, et al, 2007, 1125-1143; H. L. Kee, et al., 2007, 1110-1124], (b) installation of an additional ring on the periphery of the macromolecule (exocyclic ring) [J. K. Laha, et al., 2006], or (c) assembling of the chlorins and bacteriochlorins into strongly conjugated arrays, i.e., arrays where two macrocycles are connected by a linker that provides strong 7-conjugation between subunits [Z. Yu, et al., 2014].

“Antibody” and “antibodies” as used herein refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies, or domain antibodies (dAbs), and including single domain antibodies sdAbs that are naturally occurring, e.g., as in cartilaginous fishes and camelid, or which are synthetic, e.g., nanobodies, and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2), or subclass.

“Antibody fragment” as used herein refers to a portion of an intact antibody comprising the antigen-binding site or variable region. The portion does not include the constant heavy chain domains (i.e., CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)₂ fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

As defined herein, a “receptor” includes any bioentity which can bind small molecules, a peptide, a protein, DNA, and/or a sugar.

I. NIR-Emitting Pdots and Methods of Making Same

Broadly, in a first aspect, NIR-emitting Pdots are described herein, wherein the NIR-emitting Pdots comprise at least one semiconducting polymer and at least one doping dye. In some embodiments, the synthesis of the NIR-emitting Pdots of the first aspect is described. In some embodiments, the NIR-emitting Pdots are prepared by doping at least one semiconducting polymer (e.g., poly[(9,9-dioctylfluorenyl-2,7-diyl)-alt-co-(1,4-benzo-(2,1′,3)-thiadiazole)] (PFBT), a commonly used host polymer in luminescent Pdots) with at least one doping dye.

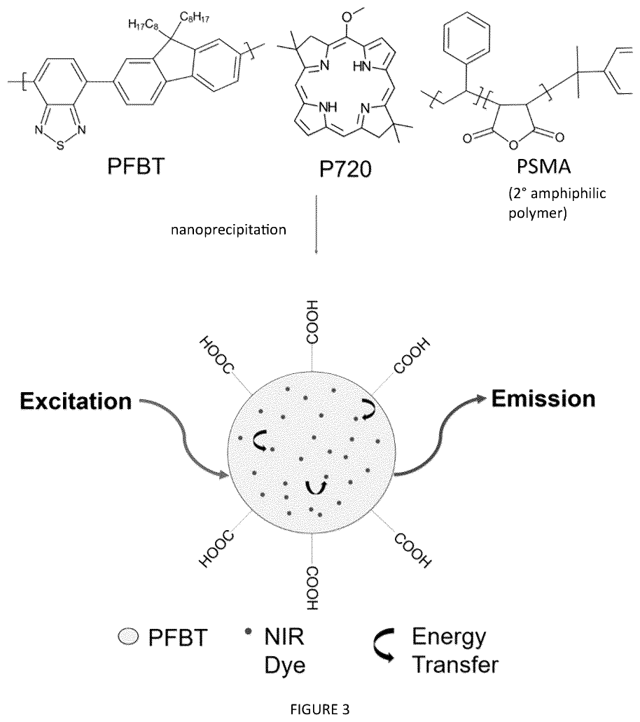

A schematic diagram which describes the nanoprecipitation reaction to form NIR-emitting Pdots of the first aspect is shown in FIG. 3. In this method, the precursor materials including at least one semiconducting polymer (e.g., PFBT), optionally at least one additional component to impart Pdots solubility in aqueous solutions (e.g., Poly(styrene/maleic anhydride (PSMA)), and at least one doping dye are dissolved in at least one water-miscible organic solvent including, but not limited to, tetrahydrofuran (THF), ethanol, and combinations thereof to produce a reaction mixture. A sample of this reaction mixture is then rapidly injected into a larger volume of water and sonicated using a standard commercial laboratory sonicator. The hydrophobic nature of the semiconducting polymers drives their aggregation into substantially spherical particles to minimize hydrophobic interactions. The organic solvent (e.g., THF) is then removed, and the particles filtered to remove any large aggregates. In some embodiments, the method of forming NIR-emitting Pdots can take as little as 10 min and requires no specialized equipment.

Accordingly, in some embodiments, a method of making near infrared (NIR)-emitting polymer dots (Pdots) is described, said method comprising:
dissolving at least one semiconducting polymer, optionally at least one additional component, and at least one doping dye in at least one water-miscible organic solvent to form a reaction mixture; and
coprecipitating the at least one semiconducting polymer, optionally at least one additional component, and at least one doping dye to form NIR-emitting Pdots by injecting the reaction mixture into a volume of water under sonication.
   In some embodiments, the method further comprises removing the at least one organic solvent and filtering the NIR-emitting dots to remove any large aggregates. In some embodiments, the at least one organic solvent comprises, consists of, or consists essentially of, THF.

In some embodiments, the at least one semiconducting polymer includes, but is not limited to, PFBT, Poly[9,9-dioctylfluorenyl-2,7-diyl] (PFO), and Poly [2-(5-cyano-methylhexyloxy)-1-4-phenylene] (CNPPP). In some embodiments, the at least one semiconducting polymer comprises PFBT. In some embodiments, the at least one semiconducting polymer comprises PFO. In some embodiments, the at least one semiconducting polymer comprises CNPPP.

In some embodiments, the concentration of doping dyes in the reaction mixture is in a range from about 1 wt % to about 10 wt %, or about 2 wt % to about 8 wt %, or about 3 wt % to about 7 wt %, or about 3 wt % to about 6 wt %, based on the total weight of the reaction mixture.

In some embodiments, the at least one additional component can be included to alter the Pdots surface and optical properties. In some embodiments, the at least one additional component is combined with the at least one semiconducting polymer and the at least one doping dye prior to injection into water and sonication (e.g., before Pdots formation). In some embodiments, the at least one additional component is added to the Pdots after Pdots formation, for example, to modify the Pdots. In some embodiments, the at least one additional component includes, but is not limited to, an amphiphilic surfactant, an electrolyte, a lipid, and/or dextran, which is/are included to increase solubility in aqueous solutions, increase stability, and/or provide binding sites for future functionalization. In some embodiments, the at least one additional component comprising an amphiphilic surfactant, an electrolyte, a lipid, and/or dextran is added before Pdots formation. In some embodiments, the amphiphilic surfactant includes PSMA, a poly(styrene) co-polymer with carboxylated poly (ethylene glycol) (PS-PEG-COOH) and/or maleic anhydride. The hydrophobic end of the surfactant is incorporated into the polymer matrix during the formation of the Pdots, while the hydrophilic end remains on the surface, providing electrostatic and steric repulsion to reduce aggregation, as well as functional groups for further functionalization. In some embodiments, the lipid includes, but is not limited to, 1,2-distearoyl-sn-glycero-3-phosphoethanolamine (DSPE), 1,2-distearoyl-sn-glycero-3-phosphoethanolamine-PEG-carboxylic acid (DSPE-PEG-COOH) and dioleoyl-3-trimethylammonium propane (DOTAP). In some embodiments, the at least one additional component comprises an antibody, which is added after Pdots formation.

In some embodiments, the NIR-emitting Pdots are substantially spherical with an average diameter of about 30 to about 65 nm, or about 35 to about 60 nm, or about 40 to about 55 nm, or about 40 to about 50 nm, or about 45 to about 55 nm, as determined using TEM.

In some embodiments, the doping dye is substantially encapsulated or embedded in the polymer matrix. In some embodiments, the doping dye is covalently bound to the semiconducting polymer itself. In some embodiments, a portion of the doping dye is both encapsulated/embedded in the polymer matrix and a portion is covalently bound to the semiconductor polymer.

Advantageously, the efficient energy transfer between the semiconducting polymer (e.g., PFBT) and the doping dyes (e.g., chlorins or bacteriochlorins) decreases the semiconducting polymer donor emission to near baseline level and increases the emission of the doping dyes that serve as acceptors. Further, the NIR-emitting Pdots of the first aspect are water-soluble nanostructures that feature a common excitation wavelength, characteristic for the polymer, in the visible region with tunable, narrow deep-red/near-IR emissions from the doping dyes.

a. Hydroporphyrin-Doping Dyes

In some embodiments of the first aspect, the doping material comprises a series of hydroporphyrins (e.g., chlorins and bacteriochlorins) having varying auxochromes, including, but not limited to, phenyl, ethynyl, phenylethynyl, vinyl, styryl, acetyl, alkoxycarbonyl, carboxylate, hydroxy, cyano, amino and combination thereof. Hydroporphyrins such as chlorins and bacteriochlorins are ideal dopants due to their high hydrophobicity, which precludes their use as molecular probes in aqueous biological media while at the same time preventing their leakage when doped into Pdots. Additionally, chlorins and bacteriochlorins have narrow deep red to NIR-emission bands and the wide array of synthetic modifications available for modifying their molecular structure enables tuning their emission predictably and systematically.

Accordingly, in some embodiments, the NIR-emitting Pdots comprise at least one semiconducting polymer (e.g., PFBT), and at least one hydroporphyrin doping dye. In some other embodiments, the NIR-emitting Pdots comprise at least one semiconducting polymer (e.g., PFBT), at least one additional component (e.g., PSMA), and at least one hydroporphyrin doping dye.

In some embodiments, the hydroporphyrin doping dye is selected from one or more of: phthalocyanines such as P700; red-emitting chlorins selected from one or more of P660, P640, and P690; near-IR-emitting bacteriochlorins selected from one or more of P720, P790 and P820; chlorin-chlorin multimers with conjugated and cross-conjugated linkers such as ethynyl, butadienyl, ethenyl, and oxo; bacteriochlorin-bacteriochlorin multimers with conjugated and cross-conjugated linkers such as ethynyl, butadienyl, ethenyl, and oxo; chlorin-bacteriochlorin multimers with conjugated and cross-conjugated linkers such as ethynyl, butadienyl, ethenyl, and oxo; non-conjugated chlorin-chlorin multimers with linkers such as ester, amide, ether, phosphonate, phosphate, and amine; non-conjugated bacteriochlorin-bacteriochlorin with linkers such as ester, amide, ether, phosphonate, phosphate, and amine; non-conjugated chlorin-bacteriochlorin multimers with linkers such as ester, amide, ether, phosphonate, phosphate, and amine; and any combination thereof. In some embodiments, the at least one hydroporphyrin doping dye comprises a red-emitting chlorin. In some embodiments, the at least one hydroporphyrin doping dye comprises a red-emitting chlorin selected from one or more of P660, P640, and P690. In some embodiments, the at least one hydroporphyrin doping dye comprises a near-IR-emitting bacteriochlorin. In some embodiments, the at least one hydroporphyrin doping dye comprises a near-IR-emitting bacteriochlorin selected from one or more of P720, P790 and P820.

In some embodiments, the NIR-emitting Pdots comprising hydroporphyrin doping dyes can be excited by a single absorption/excitation wavelength and show narrow emission wavelengths ranging from about 640 to about 820 nm depending on the specific doping dye in the doped Pdots. In some embodiments, the single excitation wavelength corresponds to that of the peak absorption wavelength of at least one semiconducting polymer. In some embodiments, the at least one semiconducting polymer comprises PFBT and the single excitation wavelength is about 450 nm. In some embodiments, the doping dye is chosen to tune the peak emission wavelength to somewhere between 640 to about 820 nm.

In some embodiments, the NIR-emitting Pdots comprising hydroporphyrin doping dyes have an emission quantum yield in a range from about 0.11 to about 0.49.

In some embodiments, the NIR-emitting Pdots comprising hydroporphyrin doping dyes have a surface that is functionalized with negatively charged carboxyl groups. In some embodiments, the NIR-emitting Pdots comprising hydroporphyrin doping dyes have a surface that is functionalized with negatively charged carboxyl groups, as evidenced by the zeta potential. In some embodiments, Pdots functionalized by carboxyl groups have a zeta potential less than about −30 mV.

b. Benzo-Bis-Thiadiazole (BBTD) Dyes

In some embodiments of the first aspect, the doping dye comprises at least one BBTD dye having varying functional groups including, but not limited to, pyrrole, thiophene, furan, N,N-dimethylamine, N,N-diphenylamine, julolidine, and any combination thereof, which are characterized with longer emission wavelengths than porphyrins. Advantageously, the NIR-emitting Pdots comprising BBTD dyes have an emission wavelength range between about 780 nm to about 1000 nm, which greatly expands the wavelength range of the NIR-emitting Pdots described herein.

Accordingly, in some embodiments, the NIR-emitting Pdots comprise at least one semiconducting polymer (e.g., PFBT), and at least one BBTD doping dye. In some other embodiments, the NIR-emitting Pdots comprise at least one semiconducting polymer (e.g., PFBT), at least one additional component (e.g., PSMA), and at least one BBTD doping dye.

In some embodiments, the BBTD doping dye is 4,8-Dibromobenzo[1,2-c;4,5-c′]bis[1,2,5]thiadiazole (BBTD-Br₂) or a derivative thereof. In some embodiments, the BBTD doping dye is selected from one or more of BBTD-Br₂, BBTD850, BBTD990, and BBTD780. In some other embodiments, the BBTD doping dye comprises the benzobis[1,2,5]thiadiazole moiety.

In some embodiments, the NIR-emitting Pdots comprising BBTD doping dyes can be excited by a single absorption/excitation wavelength and show emission wavelengths ranging from about 700 to about 1000 nm depending on the specific doping dye in the doped Pdots. In some embodiments, the single excitation wavelength corresponds to that of the peak absorption wavelength of at least one semiconducting polymer. In some embodiments, the at least one semiconducting polymer comprises PFBT and the single excitation wavelength is about 450 nm. In some embodiments, the doping dye is chosen to tune the peak emission wavelength to somewhere between 700 to about 1000 nm.

II. Antibody-Pdots Conjugates

Due to their powerful optical properties, Pdots have been investigated as probes for fluorescence imaging. In some embodiments, for specific cellular imaging, the surface of Pdots can be modified by coupling the carboxyl groups on the surface of the Pdots (see, e.g., FIG. 3) to a cellular targeting moiety, for example an antibody towards a cellular target, using an N-(3-Dimethylaminopropyl)-NO-ethylcarbodiimide (EDC) coupling reaction. The high per-particle brightness, resistance to photobleaching, selective imaging capabilities of Pdots-antibody conjugates, and the inherent photoblinking properties of Pdots make them ideal candidates for wide field, confocal and super-resolution cellular imaging. To the best of the present inventors' knowledge, as of filing, the highest reported resolution using Pdots is 57 nm, several times smaller than the diffraction limit of light [Z. Liu, et al., 2020].

Accordingly, in a second aspect, a conjugate comprising a NIR-emitting Pdot of the first aspect and an antibody is described. In some embodiments, the antibody-Pdots conjugate is obtained by coupling one or more antibodies with one or more NIR-emitting Pdots of the first aspect using an EDC coupling reaction. In some embodiments, a method of obtaining an antibody-Pdots conjugate of the second aspect is described, said method comprising mixing an amount of a solution comprising NIR-emitting Pdots of the first aspect with a poly(ethylene) glycol (PEG) solution and a buffer (e.g., a HEPES buffer). To this mixture, an amount of a solution comprising an antibody is added and stirred to ensure complete mixing. Then an amount of EDC is added and the mixture agitated to effectuate the coupling reaction to conjugate or functionalize the NIR-emitting Pdots with the antibody.

In some embodiments, a method of making an antibody-Pdots conjugate is described, said method comprising:
mixing an amount of a solution comprising NIR-emitting Pdots with a poly(ethylene) glycol (PEG) solution and a buffer to form a first mixture;
adding a solution comprising an antibody to the first mixture with mixing to form a second solution; and
adding an amount of N-(3-Dimethylaminopropyl)-NO-ethylcarbodiimide (EDC) with agitation to effectuate a coupling reaction to conjugate the NIR-emitting Pdots with the antibody.
   In some embodiments, the NIR-emitting Pdots comprise the NIR-emitting Pdots of the first aspect.

In some embodiments, the antibody-Pdots conjugate of the second aspect have a lower negative surface charge than the NIR-emitting Pdots of the first aspect, having the same doping dye, due to antibody conjugation which decrease the number of negatively charged carboxyl groups on the Pdots' surface.

In some embodiments, the antibody-Pdots conjugate of the second aspect comprises a secondary antibody, wherein binding of secondary antibody-conjugated Pdots to primary antibody-labeled receptors in or on cells can be effectuated for qualitative or quantitative fluorescence imaging.

III. The Use of NIR-Emitting Pdots as Fluorescent Probes

Expanding Pdots imaging and sensing capabilities to the near infrared is important to successfully image or sense molecular targets in complex biological matrices that show high levels of autofluorescence and light scattering. In such matrices, for example plant cells and tissue, the ability to image molecular targets in the visible region of the electromagnetic spectrum is greatly impaired. Advantageously, cellular and tissue autofluorescence and light scattering present minimal or no interference with fluorescence signals of NIR-emitting Pdots of the first aspect (or conjugates of the second aspect) despite the use of a visible excitation wavelength.

Accordingly, in a third aspect, the NIR-emitting Pdots of the first aspect can be used for multiplex cellular fluorescence imaging of one or more targets including, but not limited to, different receptors, cells, tumors, and lymph nodes. In some embodiments, the target is on the cell membrane. In some embodiments, the target is in a cell. In one embodiment of the third aspect, a method of imaging one or more targets in a cell or on a cell membrane using fluorescence imaging is described, said method comprising incubating a mixture comprising one or more NIR-emitting Pdots of the first aspect with the cells for a time to effectuate incorporation of the one or more NIR-emitting Pdots of the first aspect into the cell or onto the cell membrane, and imaging said cells using fluorescence measurement means. In some embodiments of the third aspect, a method of using NIR-emitting Pdots for enhanced fluorescence imaging of one or more targets comprises incubating a mixture comprising one or more NIR-emitting Pdots for a time to effectuate incorporation of the one or more NIR-emitting Pdots into and/or onto the target, and imaging said target using fluorescence measurement means. In some embodiments, the cells are incubated and imaged in vitro, in vivo, or ex vivo. In some embodiments, the fluorescence imaging is qualitative to obtain an image of the target. In some other embodiments, the method is intended to be quantitative, wherein the fluorescence intensity can be compared with the fluorescence intensity of a control of known concentration, to determine a concentration or amount of the target in the cell or on the cell membrane. In some embodiments, the one or more NIR-emitting Pdots can be excited by a single absorption/excitation wavelength in the visible region and the detected emission wavelengths range from about 640 to about 1000 nm depending on the specific doping dye in the doped Pdots. In some embodiments, wide-field fluorescence microscopy is used. In some embodiments, the cells are plant cells. In some embodiments, the cells are tissue cells. In some embodiments, the one or more targets have high levels of autofluorescence and light scattering, and as such, the one or more targets can only be successfully imaged using, for example, the NIR-emitting Pdots of the first aspect. Advantageously, the method of the third aspect exhibits minimal or no cytotoxicity towards the cells.

In a fourth aspect, a method for targeted imaging of receptors in or on cells using the antibody-Pdots conjugate of the second aspect is described. In some embodiments, the target is on the cell membrane. In some embodiments, the target is in the cells. In one embodiment of the fourth aspect, a method for targeted imaging of receptors in a cell or on a cell membrane using fluorescence imaging is described, said method comprising incubating a mixture comprising one or more secondary antibody-Pdots conjugates of the second aspect with the cells for a time to effectuate labeling of the secondary antibody-conjugated Pdots to primary antibody-labeled receptors in or on cells, and imaging said labeled cells using fluorescence measurement means. In some embodiments of the fourth aspect, a method of using antibody-Pdots conjugates for enhanced fluorescence imaging of one or more targets comprises incubating a mixture comprising one or more secondary antibody-Pdots conjugates for a time to effectuate labeling of the one or more secondary antibody-Pdots conjugates to primary antibody-labeled receptors associated with the target, and imaging said target using fluorescence measurement means. In some embodiments, the cells are labeled and imaged in vitro, in vivo, or ex vivo. In some embodiments, the fluorescence imaging is qualitative to obtain an image of the receptors in or on cells. In some other embodiments, the method is intended to be quantitative, wherein the fluorescence intensity can be compared with the fluorescence intensity of a control of known concentration, to determine an amount of receptors available in or on the cell. In some embodiments, the one or more NIR-emitting Pdots of the antibody-Pdots conjugate can be excited by a single absorption/excitation wavelength in the visible region and the detected emission wavelengths range from about 640 to about 1000 nm depending on the specific doping dye in the doped Pdots. In some embodiments, wide-field fluorescence microscopy is used. In some embodiments, the cells are plant cells. In some embodiments, the cells are tissue cells. In some embodiments, the receptors include, but are not limited to, FLAG-tagged FLS-2 membrane receptors. Advantageously, the method of the fourth aspect exhibits minimal or no cytotoxicity towards the cells.

In addition to plant cells, a variety of mammalian cell types may be imaged using the methods of the third or fourth aspect. Such cell types can be found in tissues and fluid including in biopsy and autopsy samples, oropharyngeal specimens, nasopharyngeal specimens, frozen sections taken for histologic purposes, blood (such as whole blood, dried blood spots, etc.), plasma, serum, red blood cells, platelets, interstitial fluid, cerebrospinal fluid, etc. The cell types may be found in lymph fluid, cerebrospinal fluid, or any fluid collected by aspiration. A cell type may be provided by removing a sample of cells from a human or a non-human animal, but also can include previously isolated cells (e.g., isolated by another person, at another time, and/or for another purpose). Cells present in archival tissues, such as those having treatment or outcome history, may also be used. The cells can be intact, hydrated, live, or dead.

In a fifth aspect, the NIR-emitting Pdots of the first aspect are used to visualize networks in an object (e.g., a mammalian body or a plant), for example lymphatic networks and lymph nodes. Advantageously, by performing simultaneous color lymphatic imaging using one or more NIR-emitting Pdots of the first aspect described herein, it is possible to determine the optimal lymph node dissection. In some embodiments, the visualization of lymphatic networks comprises administering one or more NIR-emitting Pdots of the first aspect to the lymphatic network, exciting the one or more NIR-emitting Pdots with visible radiation, and measuring the emissions from the one or more NIR-emitting Pdots to visualize the lymphatic network. In some embodiments, more than one NIR-emitting Pdots of the first aspect are administered and the emissions are simultaneous and multi-colored. In some embodiments, the sentinel node metastasis can be identified once lymphatic networks are visualized.

In a sixth aspect, the hydroporphyrin-doped Pdots of the first aspect show a blinking behavior that enable their use in super-resolution imaging methods like STORM or PALM.

As described herein, the methods of imaging or visualization described herein in the third-sixth aspects are limited to the use of the NIR-emitting Pdots (of the first aspect) or the antibody-Pdots conjugates (of the second aspect). That said, it should be appreciated by the skilled artisan that other emitting species, whether commercial or not, can be included with the NIR-emitting Pdots (of the first aspect) or the antibody-Pdots conjugates (of the second aspect) during the methods of imaging or visualization described herein.

IV. Oxygen Sensing Pdots

In a seventh aspect, oxygen-sensing Pdots based on the dye tris (4,7-diphenyl-1,10-phenanthroline) ruthenium (II) dichloride, Ru(dpp)₃²⁺ are described herein. In some embodiments, the method of making the oxygen-sensing Pdots of the seventh aspect comprises combining a blue-emitting polymer (e.g., PFO), optionally at least one additional component (e.g., Poly(styrene/maleic anhydride (PSMA)), and the dye tris (4,7-diphenyl-1,10-phenanthroline) ruthenium (II) dichloride, Ru(dpp)₃²⁺, which are dissolved in an ethanol/THF mixture (e.g., a 10% ethanol/THF mixture). A sample of this solution is then rapidly injected into a larger volume of water under vigorous sonication. The organic solvent (e.g., ethanol/THF) is then removed, and the particles filtered to remove any large aggregates. Despite the dye being partially water soluble, the dye molecules were encapsulated in the Pdots with high efficiency and the oxygen-sensing Pdots show no measurable dye leakage in aqueous buffers.

Accordingly, in some embodiments of the seventh aspect, a method of making oxygen-sensing Pdots is described, said method comprising:
dissolving at least one semiconducting polymer, optionally at least one additional component, and the dye tris (4,7-diphenyl-1,10-phenanthroline) ruthenium (II) dichloride in at least one water-miscible organic solvent to form a reaction mixture; and
coprecipitating the at least one semiconducting polymer, optionally at least one additional component, and Ru(dpp)₃²⁺ to form oxygen-sensing Pdots by injecting the reaction mixture into a volume of water under sonication.
   In some embodiments, the method further comprises removing the at least one organic solvent and filtering the oxygen-sensing dots to remove any large aggregates. In some embodiments, the at least one organic solvent comprises a mixture of THF and ethanol.

In forming oxygen-sensitive Pdots, the blue-emitting PFO polymer was used for better overlap with the absorption spectrum of Ru(dpp)₃²⁺. Advantageously, Ru(dpp)₃²⁺ shows significant fluorescence enhancement when doped in the Pdot compared to the dye alone. In some embodiments, the fluorescence enhancement of Ru(dpp)₃²⁺ doped in a Pdot is about 25 to about 35 times greater than that of the dye alone.

Accordingly, in some embodiments of the seventh invention, oxygen-sensing Pdots comprising tris (4,7-diphenyl-1,10-phenanthroline) ruthenium (II) dichloride Ru(dpp)₃²⁺ and PFO. In some other embodiments, oxygen-sensing Pdots comprising tris (4,7-diphenyl-1,10-phenanthroline) ruthenium (II) dichloride Ru(dpp)₃²⁺+, PFO, and at least one additional component are described. In some other embodiments, oxygen-sensing Pdots comprising tris (4,7-diphenyl-1,10-phenanthroline) ruthenium (II) dichloride Ru(dpp)₃²⁺, PFO, and PSMA are described.

In some embodiments of the seventh aspect, the oxygen-sensing Pdots are used to measure the level of oxygen in an environment using fluorescence imaging. In some embodiments, the oxygen-sensing Pdots are used as a hypoxia sensor in a cell.

### Example 1

### Experimental Section

### 1.1. Chemical Reagents

Poly [(9,9-dioctylfluorenyl-2,7-diyl)-alt-co-(1,4-benzo-(2,1′,3)-thiadiazole)] (PFBT) (37 000 MW, 3.3 polydispersity), poly [2-methoxy-5-(3,7-dimethyl-octyloxy)-1,4-phenylenevinylene] (ADS104) (100 000 MW, 3.3 polydispersity), poly[{9,9-dihexyl-2,7-bis(1-cyanovinylene)fluorenylene}-alt-co-{2,5-bis(N,N′-diphenylamino)-1,4-phenylene}] (ADS111) (48 000 MW, 2.9 polydispersity), poly[{2-methoxy-5-(2-ethylhexyloxy)-1,4-(1-cyanovinylenephenylene)}-alt-co-{2,5-bis(N,N′-diphenylamino)-1,4-phenylene}] (ADS113) (40 000 MW, 3.5 polydispersity), and Poly[9,9-dioctylfluorenyl-2,7-diyl]-end capped with Dimethylphenyl (DMP) were purchased from American Dye Source, Baie-D'Urfe, QC, Canada. Poly(styrene/maleic anhydride) (PSMA) [67:33] (MW 7500) was purchased from Polysciences, Warrington, PA, USA. Tetrahydrofuran (THF, anhydrous, >99.8%), phosphate-buffered saline (PBS), and bovine serum albumin (BSA) were purchased from Fisher Scientific, Hampton, NH, USA. Dulbecco's modified Eagle medium, fetal bovine serum, HEPES buffer, and sodium pyruvate were purchased from ThermoFisher Scientific, Waltham, MA, USA. Rat endothelial cells (RECs) were previously isolated in the lab. Anti-Rabbit IgG (H+L), F(ab) fragment antibody produced in goat, N-(3-dimethylaminopropyl)-N′-ethylcarbodiimide (EDC), Tris-HCl, NaCl, Driselase, MES, Poly(ethylene glycol) 3350 MW, Sodium Azide, Tris(4,7-diphenyl-1,10-phenanthroline) ruthenium (II) dichloride, skim-milk powder, Tween 20, and Triton X-100 were purchased from Millipore Sigma, St. Louis, MO, USA. The GST-tag antibody [HRP] produced in rabbits was purchased from GenScript, Piscataway, NJ, USA. The nitrocellulose membrane and the Western blot chemiluminescent substrate kit were purchased from Fisher Scientific, Hampton, NH, USA. Paraformaldehyde (PFA) was purchased from Electron Microscope Sciences, Hatfield, PA, USA. The Murisage and Skoog mixture was purchased from Caisson, Smithfield, UT, USA. The DYKDDDDK Tag Recombinant Rabbit monoclonal antibody was purchased from Invitrogen, Waltham, MA, USA. All commercial materials were used as received.

### 1.2. Synthesis and Characterization of NIR Porphyrin Dyes

Specifically, the synthesis protocols for red-emitting chlorins P660, P640, and P690 as well as near-IR-emitting bacteriochlorins P720 and P820 were reported previously. The synthesis of P790 is described in detail in the [C. Riahin, et al., 2022, Supporting Information]. The notation PXXX stands for porphyrin (P) (chlorin and bacteriochlorins are hydroporphyrins) and the dye's emission peak wavelength. Microwave reactions were performed in a CEM Discover (CEM, Mathew, NC) microwave instrument. All reactions were performed in a 10 mL CEM pressurized microwave vessel, with continuous monitoring of pressure and temperature. The temperature was monitored using a built-in IR sensor. All NMR spectra were acquired on a 400 MHz JOEL ECX-400 NMR.

### 1.3. Polymer Dots (Pdots): Synthesis and Characterization

Porphyrin-doped Pdots were synthesized via a nanoprecipitation method [Y. Jin, et al., 2011]. PFBT, PSMA, and the porphyrin dyes were dissolved in anhydrous THF overnight, each in separate 25 mL round-bottom flasks. Solutions were stored under a nitrogen atmosphere at room temperature, with the dyes stored away from light. The three precursors were filtered through a 0.22 μm PTFE syringe filter and then mixed together in 1 mL of THF. The concentrations of each of the precursors in the mixture were 100 ppm (PFBT), 100 ppm (PSMA), and 2-20 ppm (dye) depending on the dye. The solution was then injected into 10 mL of DI water under vigorous sonication at room temperature for 1 min. THF was removed through vacuum evaporation on a rotary evaporator at 60° C. All samples were filtered through a 0.22 μm cellulose acetate syringe filter prior to further studies.

The size and morphology of the synthesized Pdots were measured using an FEI Morgagni 268 100 kV TEM, Morgangi, Hillsboro, OR, USA. Samples were prepared by placing a drop of Pdot solution on a Ted Pella (Redding, CA, USA) copper-supported grid and drying at room temperature. Hydrodynamic size and surface ζ-potential were measured with a DTS1070 folded capillary cell in a Malvern Zetasizer Nano (Model No. ZEN3600) instrument. UV-vis absorption spectra of the Pdots in aqueous solution were measured with an Aligent Cary UV-Vis multicellular Peltier spectrophotometer (Model No. G9864A). Fluorescence spectra of the dye-doped Pdots were measured using a Photon Technology International fluorimeter or a Horiba Quantamasrter 400 fluorimeter, Kyoto, Japan. All samples were measured in DI water and excited at 450 nm. Corrected spectra were collected from 475 to 850 nm. Fluorescence quantum yields were measured in air-equilibrated solvents using tetraphenylporphyrin (TPP) in air-equilibrated toluene (Φ_{f}=0.070)^{DH} as a standard. Fluorescence microscopy experiments were performed using an Olympus ix73 fluorescence microscope equipped with multiple ports. Fluorescence and bright-field images were captured with a Hamamatsu ORCA-Flash4.0 digital CMOS camera. Samples were illuminated by a CoolLED pE-300^{ultra} microscope illuminator. The typical exposure time was 300 ms. For fluorescence spectroscopy images, a Semrock QDLP-B-000 filter cube containing an FF01-435/40-25 excitation filter, an FF01-500/LP-25 emission filter, and an FF510-Di02-25×36 dichroic filter was utilized. Sample spectra of selected areas were captured using an Andor Shamrock SR303i spectrograph equipped with a 150 L/MM-500 NM grating. The spectra were acquired using a Zyla 4.2 sCMOS camera.

### 1.4. Conjugation of Secondary Antibodies to Pdots

IgG secondary antibody conjugation was achieved through a previously reported method [D. Wang, et al., 2020]. A 1 mL aliquot of 100 μg/mL Pdot solution was mixed with 20 μL of 5 wt % PEG solution (3350 MW) and 20 μL of 1 M HEPES buffer. To this mixture, 10 μL of 2 mg/mL IgG secondary antibody solution was added and the mixture stirred in a vortex to ensure complete mixing. Finally, 20 μL of 5 mg/mL N-(3-dimethylaminopropyl)-N′-ethylcarbodiimide was added and the mixture was placed on a rotary shaker for 2 h. Afterward, the mixture was transferred to a 100 kDa centrifugal filter unit alongside 10 μL of 10 wt % Triton X-100 and centrifuged at 5000 RCF for 10 min to remove the unbound antibody. Conjugation was confirmed using dynamic light scattering and ζ-potential measurements and Dot Blot. In Dot Blot experiments, a Goat Anti-Rabbit IgG secondary antibody (original 2 mg/mL, used as the positive control), pure Pdots (used as negative control), and antibody-conjugated Pdots were diluted by 10-fold multiple times, and 2 μL of each sample was spotted onto the nitrocellulose membrane. After blocking nonspecific sites by soaking in 5% skim milk powder dissolved in TBST buffer (20 mM Tris-HCl pH 7.5, 150 mM NaCl, 0.1% Tween 20) for 0.5-1 h, the nitrocellulose membrane was incubated with the primary antibody conjugated with HRP (GST-tag antibody [HRP] produced in rabbit) for 1 h at RT. Afterward, the nitrocellulose membrane was washed three times with TBST buffer (3×5 min) and incubated with the Western blot chemiluminescent substrate for 1 min and then was used to expose X-ray films in the dark room.

### 1.5. Cellular Imaging and Toxicity Studies

Rat endothelial cells cultured in Dulbecco's modified Eagle medium (DMEM) high glucose supplemented with 10% FBS, 1% HEPES, and 1% sodium pyruvate were used in the cellular imaging studies. Cell viability measurements were carried out by seeding ˜50 000 cells in each well of a 24-well plate and allowing the cells to grow for 24 h. The cells were then washed twice with phenol red- and serum-free DMEM. Pdots (˜10 and 50 μg/mL) were then added to the cells after being filtered and diluted in phenol red-free and serum-free DMEM and incubated for 1 h. Calcein-AM and ethidium homodimer were then added to the cells at concentrations of 4 and 2 μM, respectively. For the negative control, cells were incubated with 70% ethanol to ensure complete cell death. The medium was removed from the well before imaging so the excess Pdots in solution did not interfere with the fluorescing cells, but the wells were not washed so as not to remove any dead cells. The cells were then imaged and processed using a Cytation 5 fluorescence microscope with Gen5 imaging software (Biotek). Cell quantification was performed using ImageJ, counting with the multipoint tool. Cellular imaging and spectroscopy measurements with Pdots were carried out by seeding about 100 000 cells in each well and allowing the cells to grow for 24 h. The cells were then washed twice with phenol red- and serum-free DMEM. Single-type or mixed Pdots were added to the wells and incubated with the cells for 30 min at 37° C. The cells were then washed twice with phenol red- and serum-free DMEM to remove excess Pdots not taken up by the cells. Cells were then imaged using an Olympus ix73 fluorescence microscopy system described above.

### 1.6. Construction of FLAG-FLS2 Transgenic Plants

The FLAG sequence-fused FLS2 genomic fragment was generated by a two-fragment polymerase chain reaction (PCR) approach, in which primers including the FLAG sequence were used in two separate PCR reactions. Each fragment of FLS2 was amplified by PCR from the genomic DNA of wild-type plants using gene-specific primers containing the FLAG sequence. An overlapping PCR reaction was then performed to generate the whole FLS2 genomic fragment with the FLAG sequence inserted behind the signal peptide sequence of FLS2. The PCR products were cloned into pDONR-Zeo (Invitrogen) by BP reaction and subsequently cloned into the binary vector pGWB1 (Invitrogen) by LR reaction. The construct was then transformed into Arabidopsis thalianafls2 knockout mutant plants via the Agrobacterium strain GV3101 using the “floral-dip” method. The fls2 knockout mutant was obtained from the Arabidopsis Biological Resource Center (ABRC, Ohio State University, Columbus, OH). The transgenic plants were selected by germination on a 35 μg/mL hygromycin-containing 1/2 MS medium. Homozygous T3 generation plants were used in this study. The functionality of the FLAG-tagged FLS2 receptor was confirmed by its ability to complement the phenotype of the fls2 knockout mutant.

1.7. Fluorescence Imaging of Antibody-Conjugated Pdots when Targeting the FLS2 Receptor in Plant Cells

A. thaliana seeds, expressing FLAG-tagged FLS2 (FLAG-FLS2) at the N terminus, were grown on 0.5% phytagel with Murisage and Skoog media for 10-14 days with photocycles of 16 h of light followed by 8 h of darkness. Plants were removed from the medium and fixed in 4% Paraformaldehyde for 1 h under vacuum before being stored in PBS at pH 7.2. Prior to immunostaining, a scalpel was used to slice the leaves to allow some chlorophyll release for clearer imaging. The plant cell wall was partially dissolved by 0.2% driselase in 2 mM MES at pH=5.7 at 37° C. for 15 min. The membrane was then permeabilized with 1% Triton X-100 in PBS for 20 min at room temperature. Plants were then soaked in blocking buffer (1% BSA, 0.1% tween, 2 mM sodium azide in PBS) at room temperature for 20 min. Primary Rabbit anti-DYKDDDDK-tag (anti-FLAG) antibodies were diluted to 1:250 in blocking buffer, and plants were incubated with primary antibodies overnight at 4° C. Plants were rinsed in blocking buffer three times for 20 min at room temperatures. Pdots conjugated to the secondary antibody (Goat Anti-Rabbit IgG) were then diluted to 30 μg/mL in blocking buffer and incubated for 3 h at room temperature. Plants were rinsed in PBS three times for 10 min at room temperature.

Leaves and root hairs were removed from the plant and placed in a glass-bottom dish with 10 μL of PBS under a 10 cm glass coverslip. These were imaged using an inverted fluorescence microscope (Olympus IX73). These were illuminated using a 440 nm solid-state laser (Crystalaser) at 0.1 mW. Samples were observed using a 60× magnification, water objective lens (NA 1.2, Olympus UPlanSApo). Images were collected through a 647 nm band pass filter with 57 nm width and a 710 nm band pass filter with 40 nm width. Images were captured by an EMCCD camera (Princeton Instrument Photonmax) at 0.3 s exposure time.

### 1.8. Fluorescence Imaging of Single Pdots

Pdots were diluted in water, placed on a glass coverslip, and imaged using the inverted fluorescence microscope and laser described in the previous section. Pdot samples were observed using a 100× magnification, oil immersion objective lens (NA 1.4, Olympus UPlanSApo). The laser illumination covered an area with a diameter of ˜80 μm. Emission was acquired using a band pass filter centered at 647 nm with 57 nm width.

### Results and Discussion

### 2.1. Chlorin and Bacteriochlorin, Molecular Design, Synthesis, and Characterization

Chlorins and bacteriochlorins are uniquely characterized by narrow emission bands compared to other NIR-emitting dyes with the full-width at half-maximum (FWHM) of ˜15 nm for chlorins and ˜20 nm for bacteriochlorins [J. Lindsay, 2015; M. Taniguchi, et al., 2017; E. Yang, et al., 2011; M. Taniguchi, et al., 2008]. In addition, unlike with other NIR-emitting dyes, it is possible to position and tune the emission band of chlorins and bacteriochlorins with nearly nanometer precision [Id.]. In the current study, NIR-emitting Pdots were prepared using a series of hydrophobic chlorins and bacteriochlorins (see, FIG. 1), in which all share a common hydroporphyrin macrocyclic structure. The chlorin and bacteriochlorin dyes differ in their peripheral substituents, or they are arranged into strongly conjugated arrays. The set includes a simple chlorin P640, which possesses the shortest emission wavelength, styrene-substituted P660, and a conjugated dyad P690, for which the emission wavelength is progressively bathochromically shifted. Similarly, a bacteriochlorin series that contains a simple bacteriochlorin P720, chalcone-substituted P790, and a conjugated dyad P820 were used. The full synthetic procedure to prepare P790, its NMR characterization, and UV-vis and fluorescence spectra for all dyes are shown in the Supporting Information [C. Riahin, et al., 2022].

### 2.2. Polymer Selection for NIR-Emitting Pdots

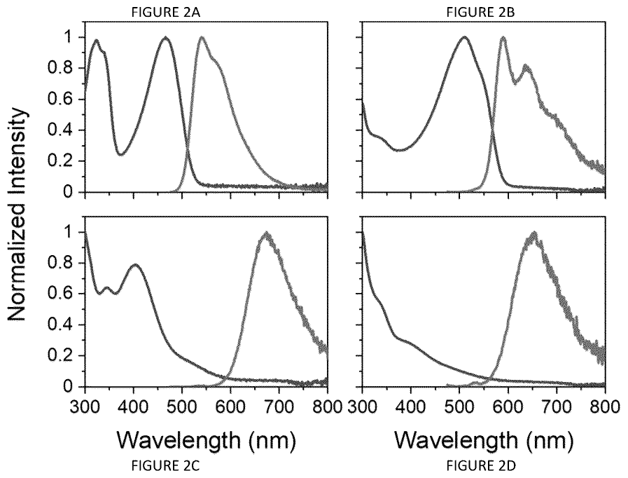

In the NIR-emitting Pdots described herein, the Pdots were excited at the peak absorption maximum of the polymer molecules. The emission maximum is obtained at the emission peak wavelength of the doped NIR dye. In some embodiments, high absorption cross section at the excitation wavelength and efficient energy transfer between the polymer and dye molecules ensure high NIR fluorescent emission of the Pdots. In some embodiments, the emission band of the polymer minimally bleeds into the emission peak of the doped dye, thereby minimizing spectral interference between the polymer and dye molecules in imaging experiments. FIGS. 2A-2D show the normalized UV-vis and fluorescence spectra of four semiconductor fluorescent polymers PFBT (FIG. 2A), ADS104 (FIG. 2B), ADS 111 (FIG. 2C), and ADS114 (FIG. 2D). The three polymers from the ADS series have been used previously in Pdots [M. Krayer, et al., 2010] but they show lower emissions than PFBT and more importantly, their emission peak tails bleed into the NIR region. PFBT was chosen as the polymer for the NIR-emitting Pdots disclosed herein because of its high absorption cross section and sufficient overlap of its fluorescence peak with the absorption peaks of the porphyrin dyes, conveniently excitable absorbance peak, and most importantly, its minimal overlap of the emission peak red tail with the dyes' emission. Using PFBT as the polymer component of the Pdots has enabled the formation of Pdots with emission peak wavelengths ranging from 640 to 820 nm. It is possible to further tune the Pdots' emission by using blue shifted-emitting polymers for visible-emitting Pdots or by using red shifted-emitting polymers like ADS104, ADS 111, and ADS113 for Pdots that emit even deeper into the NIR region.

### 2.3. Synthesis and Characterization of NIR-Emitting Pdots

Chlorin and bacteriochlorin dyes used in the present study are hydrophobic and cannot be used as molecular probes in aqueous media without incorporating them into Pdots. The synthesis of chlorin- and bacteriochlorin-doped Pdots is shown in FIG. 3. As described in the Experimental Section, the NIR-emitting Pdots were prepared by injecting PFBT, PSMA, and fluorescent dye mixture, all dissolved in THF, into aqueous solution under vigorous sonication following a previously described Pdots' synthesis protocol. Large aggregates that form during the short 1-min-long reaction are removed from the Pdots' solution by filtration.

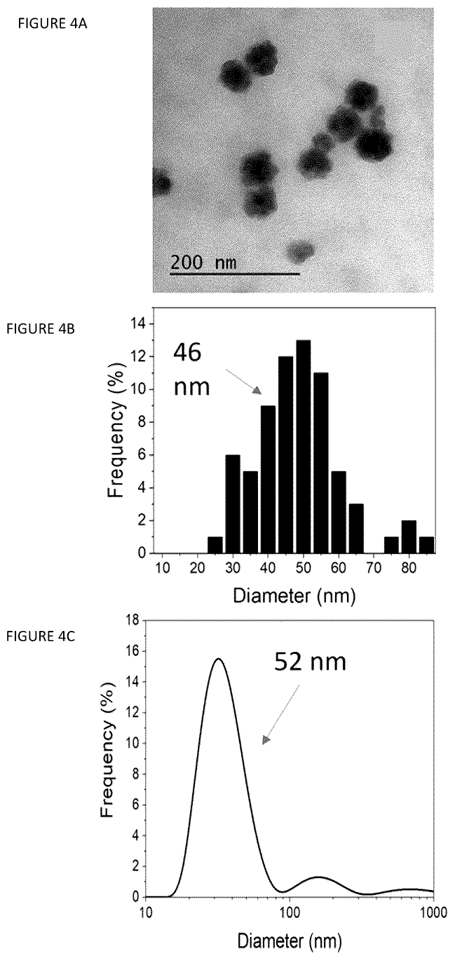

The structural properties of the formed Pdots are shown in FIG. 4. A representative TEM image (FIG. 4A) and size distribution (FIG. 4B) of the formed Pdots show substantially spherical morphology with some disorder in structure and an average diameter of 46±12 nm. While the obtained Pdots are fairly polydisperse, their emission peak wavelength is independent of their size. As expected, DLS measurements show a slightly larger average hydrodynamic diameter of 52 nm (FIG. 4C). The surface ζ-potential of the synthesized Pdots, was −37 mV, indicating that the surface is functionalized with negatively charged carboxyl groups. Although not shown, Fourier transform infrared spectroscopy shows the abundance of carboxyl groups (carbonyl peak at around 1700 cm⁻¹) when maleic anhydride groups of PSMA are transformed to carboxyl groups when the PFBT/PSMA Pdots are formed when a sample PFBT and PSMA, which are dissolved in tetrahydrofuran, is injected into an aqueous phosphate buffer solution at pH 7.2. The resulting Pdots are highly soluble in the buffer solution with no evidence of aggregation, even when stored for months in a buffer solution.

### 2.4. Pdots' NIR-Emission Properties

UV/VIS and fluorescence measurements of PFBT/PSMA Pdots in the absence of dye dopants show absorption and emission peaks at 450 and 550 nm, respectively. When the Pdots are doped with an NIR emitting dye, energy transfer between the Pdots and the doped dye molecules results in a narrow emission peak of the doped dye despite the fact that the Pdots are still excited at 450 nm. Without being bound by theory, this is believed to be due to the spectral overlap between the PFBT emission and porphyrin absorption, and the close proximity of the dyes to the polymer when doped into a Pdot, resulting in a highly efficient energy transfer from the polymer to the dye upon excitation of the porphyrin-doped PFBT Pdots at 450 nm. Advantageously, this enables the excitation Pdots, which contain different NIR emitting dyes, with the same excitation wavelength. When doped with one of the porphyrin dyes, the emission of PFBT (550 nm) is quenched and the emission shifts to that of the dye. FIG. 5 shows normalized fluorescence spectra of all six NIR-emitting Pdots. The formed porphyrin-doped Pdots feature narrow emission peaks in the NIR range with an average full-width at half-maximum (FWHM) of 25 nm. Table 1 summarizes the emission peak wavelength and emission quantum yield of the porphyrin dyes (termed PXXX) and porphyrin-doped Pdots (termed PPDXXX). An emission red shift is observed when the dyes are doped into the Pdots, most likely due to changes in their chemical environment. The magnitude of the red shift is larger at higher emission peak wavelengths. Consistent with previous studies, the emission quantum yields of the dye-doped Pdots are lower than the emission quantum yield of dye-free PFBT Pdots but the emission peaks are significantly narrower and in the NIR region. The emission quantum yields of the formed NIR-emitting Pdots range from 0.1 to 0.5 with no apparent trend. The amount of the dye dopant in the Pdots was optimized by varying the dopant concentration in the Pdots' reaction mixture to maximize their fluorescence intensity. The NIR-emission intensity of the Pdots increases with increased dye concentration up to a certain optimum level. Within this concentration range, the polymer emission is drastically reduced, and the dye emission increases. Above a certain doping percentage, the dye emission begins to decrease due to self-quenching. For example, for PPD640, a P640 concentration in the reaction mixture of 6.9 wt % resulted in the highest emission, and the NIR emission decreased above this level (data not shown). The PFBT native emission also increases when the excitation intensity is above the saturation level of the dye-doped Pdots. An example of the saturation effect is shown in FIG. 5 for PPD790 (purple curve). While energy transfer between PFBT and P790 molecules results in NIR emission at 790 nm, the emission of PFBT is only partially quenched even at a P790 level of 9.1%, the level required to maximize the PPD790 NIR emission. To prevent dye fluorescence quenching and saturation, the dye loading percentage was set to a level of 5% in all of the Pdots used in this study.

TABLE 1
Peak Emission Wavelengths and Fluorescence Quantum
Yields of Porphyrin Dyes in THF and PPDs in Water

| Sample | λₑₘ dye (nm) | λₑₘ PPD (nm) | QY dye | QY PPD |
|---|---|---|---|---|
| P640 | 637 | 641 | 0.29 ± 0.01 | 0.11 ± 0.01 |
| P660 | 657 | 662 | 0.52 ± 0.02 | 0.49 ± 0.01 |
| P690 | 686 | 692 | 0.35 ± 0.01 | 0.21 ± 0.01 |
| P720 | 711 | 718 | 0.31 ± 0.01 | 0.20 ± 0.01 |
| P790 | 778 | 794 | 0.25 ± 0.01 | 0.20 ± 0.01 |
| P820 | 800 | 821 | 0.32 ± 0.01 | 0.15 ± 0.01 |

### 2.5. Cellular Imaging and Toxicity Studies

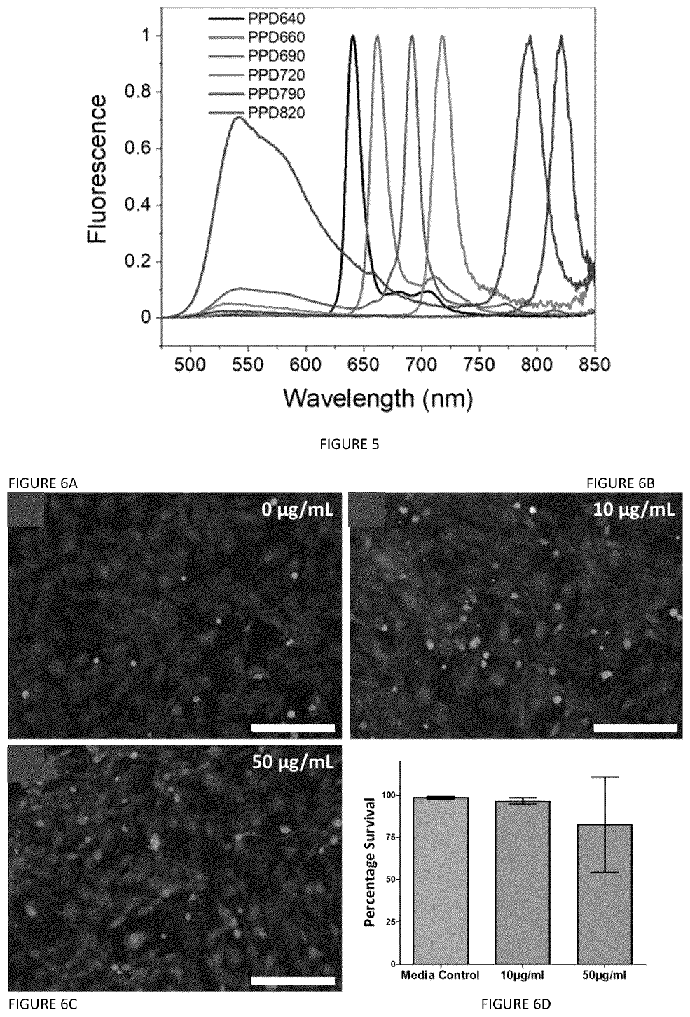

A common concern associated with the use of fluorescent nanoparticles, or in the case of the present disclosure the NIR-emitting Pdots, is whether they by themselves affect cell viability during incubation. Cell viability stain assays using Calcein-AM and ethidium homodimer-1 were carried out to determine whether the NIR-emitting Pdots described herein adversely impact cell viability upon exposure and incubation. Calcein-AM, a green fluorescent dye, is readily endocytosed by live cells; while ethidium homodimer, a red fluorescent dye, is only capable of penetrating dead cells. Fluorescence images of cells labeled with our Pdots compared to a positive control are shown in FIGS. 6A-6C, which show that cells incubated for 1 h with PPD640 concentrations 0, 10, and 50 μg/mL, respectively, had almost no fluorescence from the homodimer, indicating that most of the cells remained viable including at high concentrations of Pdots. Cell viability assays conducted following incubating the cells with NIR emitting Pdots for 24 h further support the finding that the Pdots did not adversely impact the cells when used as cellular imaging probes in cellular imaging studies.

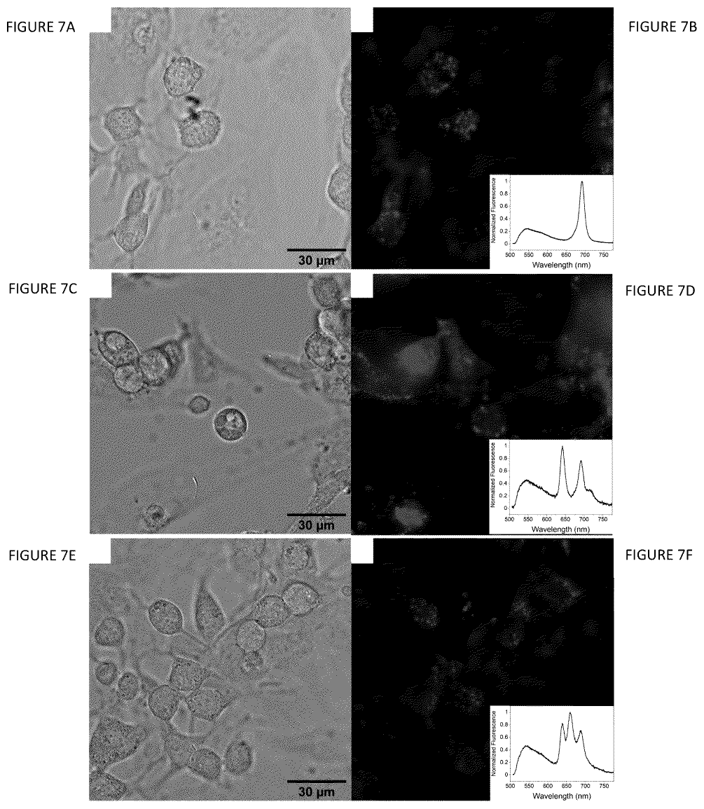

To demonstrate the multiplexing capabilities of the dye-doped Pdots, rat endothelial cells were incubated with a mixture of Pdots of varying emission wavelengths. Endothelial cells have been shown to readily endocytose nanoparticles of similar dimensions to our Pdots [P. G. Frank, et al., 2003; J. J. Li, et al., 2013]. Bright-field and fluorescence microscopy images of cells incubated with PPD640, PPD660, and PPD690 Pdots are shown in FIGS. 7A-7F. FIGS. 7A-7B show bright-field and fluorescence images of cells labeled with PPD640. The bright-field image in FIG. 7A shows that the PPD-labeled cells maintain their structural integrity. The fluorescence image in FIG. 7B shows some of the PPDs attached to the cells' membranes, while others may have permeated into the cells. The inset in FIG. 7B shows the fluorescence spectra of the subsection of the fluorescence image with high Pdot density. As expected, it shows a single fluorescence peak at 640 nm. FIGS. 7C-7D shows bright-field and fluorescence images of cells labeled with PPD640 and PPD660. The inset in FIG. 7D shows the presence of the two types of PPDs in the cells. FIG. 7E-7F shows bright-field and fluorescence images of cells labeled with PPD640, PPD660, and PPD690. The inset of FIG. 7F shows the presence of the three types of PPDs in the cells. While a baseline separation of emission peaks is not realized over the narrow emission wavelength range of about 70 nm used in these measurements, the results clearly indicate that due to their narrow emission peaks, the NIR-emitting Pdots described herein could be used for multiplex cellular fluorescence imaging of multiple targets either in the cell or on the cell membrane.

### 2.6. Fluorescence Imaging of Targeted Receptors in Plant Cells

NIR spectrum fluorescent dyes are a promising avenue for bioimaging in plants, as the presence of chlorophyll limits the types of fluorescent dyes available within the visible spectrum. The emission spectrum for Chlorophyll-b includes a peak from ˜600 to 670 nm, thus NIR dyes that emit at longer wavelengths may not be obscured by a strong chlorophyll background, making them ideal candidates for fluorescence imaging in leaves. To demonstrate the potential for the use of NIR Pdots for bioimaging in plants, Anti-Rabbit IgG (H+L), F(ab) fragment antibody molecules were conjugated to P690 containing Pdots (PPD690). The conjugation of antibody molecules to the Pdots was confirmed by dynamic light scattering (DLS), which showed an increase in the hydrodynamic diameter from 46±12 to 70±10 nm and ζ-potential from −47.8 to −34.5 mV. The activity of the antibody-Pdot conjugates was confirmed using Dot Blots, which showed a clear recognition of the primary antibody produced in rabbits by the Anti-Rabbit IgG (H+L), F(ab) fragment antibody-conjugated Pdots. The transmembrane receptor kinase FLAGELLIN SENSITIVE2 (FLS2) was stained in both leaf cells and root hair cells of A. thaliana. Both cell types express the FLS2 receptors, which play a key role in recognizing the bacterial flagellin protein and, hence, generating a defense reaction to the invading bacterial pathogen. The A. thaliana plants were genetically engineered to express a DYKDDDDK peptide, known as FLAG, tag at the FLS2 receptor N terminal. The functionality of this transgenic receptor construct was confirmed by showing that it restored flagellin recognition to A. thaliana fls2 mutant plants.

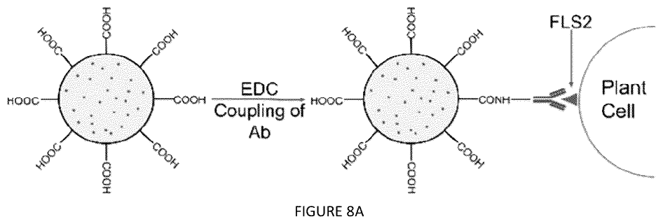

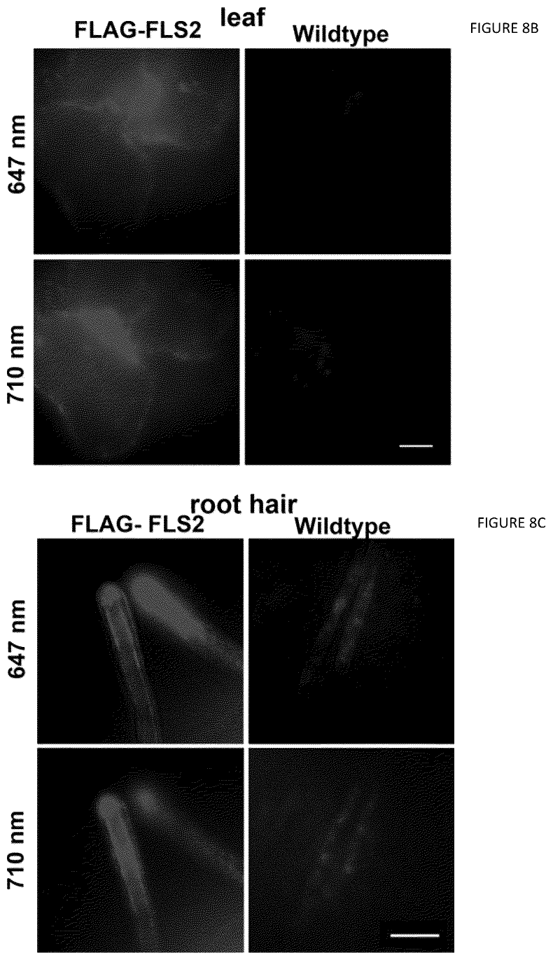

To demonstrate the utility of the Anti-Rabbit IgG (H+L), F(ab) fragment antibody-conjugated Pdots, plants expressing the FLAG-tagged FLS2 receptor were stained with a DYKDDDDK Tag Recombinant Rabbit Monoclonal Antibody. Plants were then rinsed and stained with Anti-Rabbit IgG (H +L), F(ab) fragment antibody-conjugated PPD690 Pdots. This staining was performed alongside a wild-type control plant lacking the FLAG-tagged FLS2 receptor. These plants were imaged on an inverted fluorescence microscope illuminated by a 440 nm laser in both 647 and 710 nm channels. To decrease chlorophyll interference in the leaf, small cuts were made in the leaves to allow the release of some chlorophyll, which enabled the resolution of fluorescent Pdots along the leaf cell walls in both channels (FIG. 8B, left). In contrast, no specific staining was observed in the wild type (FIG. 8B, right). The same results occurred when imaging root hair cells, showing specific fluorescence signals in both channels in the FLAG-tagged FLS2 plants but not in the wild-type plants (FIG. 8C). These results demonstrate that the antibody-coated PPD690 Pdots were able to specifically bind to FLAG-tagged receptors within fixed plant tissues. While ideally NIR Pdots in longer wavelengths will be used to circumvent the presence of chlorophyll within leaves by emitting at wavelengths beyond the chlorophyll emission spectrum, these results demonstrate as a proof of concept that antibody-conjugated Pdots within the experimental system are able to specifically recognize their target in plant tissues.

### 2.7. Fluorescence Blinking of Pdots

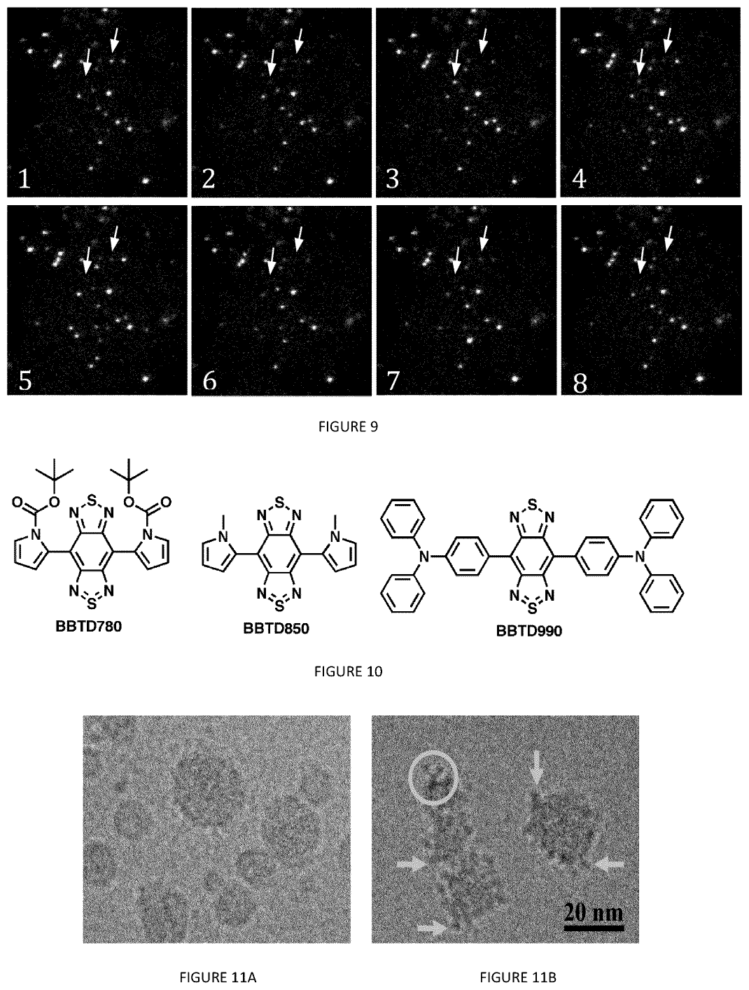

While NIR fluorescence imaging microscopy is advantageous due to the reduced autofluorescence background, its spatial resolution is diffraction-limited. Therefore, the increase in emission wavelengths decreases the spatial resolution of microscopy experiments compared to fluorescence microscopy measurements in the visible region. To overcome this spatial resolution limitation, single-particle imaging studies were conducted to determine whether the 50 nm NIR-emitting Pdots described herein could be used as super-resolution imaging probes. When an aqueous solution of PPD690 Pdots was diluted and imaged with high magnification, it was possible to capture emissions of individual Pdots as they went through on-off cycles or blinking. This blinking behavior is demonstrated in FIG. 9, where consecutive images are selected from a video (not provided herein). Such a blinking property makes these Pdots valuable for single-molecule-based super-resolution fluorescence imaging, such as STORM or PALM, where photoswitching fluorophores are required. Together with the observation that these Pdots are not toxic to living cells, their ability to blink makes them valuable for biological imaging with nm spatial resolution.

### Summary and Conclusion

The present example enables the expansion of the use of Pdots to the near infrared region, which is necessary for quantitative imaging of biological samples that are characterized with high auto fluorescence in the visible region of the electromagnetic spectrum and high light scattering like plant cells. The synthesis of deep red- and NIR-emitting Pdots that were prepared by doping Pdots with a series of chlorin and bacteriochlorin dyes with systematically tuned molecular structures was described. The resulting doped Pdots can be excited by a single excitation source and are highly emitting with distinct narrow emission peaks and high emission quantum yields over a broad range of emission wavelengths. It is normally very challenging to use chlorins and bacteriochlorins in a free form in aqueous media because of their low aqueous solubility, but advantageously doping the Pdots with chlorins and bacteriochlorins minimally alters the emission properties of the free dyes and enables their use in aqueous biological buffers. The resulting doped Pdots do not show measurable cytotoxicity or aggregation under the experimental conditions of a relatively short doped Pdot-cell exposure of 1 h. Conjugation of antibodies to Pdots is required for selective cellular imaging. When labeled with secondary antibodies, the NIR-emitting doped Pdots can be used effectively for targeted imaging of receptors in plant cells. Additionally, the data shows that the porphyrin-doped NIR-emitting Pdots photoblink. Accordingly, in some embodiments, the doped Pdots can be used as super-resolution cellular imaging probes. In some other embodiments, because chlorin- and bacteriochlorin-doped Pdots are sufficiently photostable, they can be used in wide-field fluorescence imaging applications.

### Example 2

### Experimental Section

### 1.1. Additional Chemical Reagents (in Addition to Those Disclosed in Example 1)

Palladium catalysts and thionyl chloride were obtained from Sigma-Aldrich, St. Louis, MO, USA. Diamine and boronic acids were obtained from Ambeed, Arlington Heights, IL, USA. All commercial materials were used as received.

### 1.2. Synthesis of NIR Emitting Dyes

In addition to the synthesis of the red emitting chlorins P660, P640, and P690 and the near-IR emitting bacteriochlorins P720, and P820, as described in Example 1, the red emitting chlorin P665 was also synthesized. 4,8-Dibromobenzo[1,2-c;4,5-c′]bis[1,2,5]thiadiazole BBTD-Br₂ was prepared via literature methods [M. G. Murali, et al., 2014]. BBTD-Br₂ was further derivatized by palladium catalyzed cross-coupling utilizing Schlenk technique. Known compounds BBTD850 and BBTD990 were synthesized following previously published procedures [C. Kitamura, et al., 1996; Q. Li, et al., 2020]. BBTD780 was synthesized in a similar manner using a Suzuki coupling reaction of BBTD-Br₂ with N-Boc-2-pyrroleboronic acid. A flame-dried Schlenk flask was charged with BBTD-Br₂ (150 mg, 0.426 mmol), N-Boc-2-pyrroleboronic acid (449.5 mg, 2.130 mmol), aqueous 2 M K₂CO₃ (6 mL), and THF (30 mL). The solution was then degassed 3 times by freeze-thaw-pump cycles. The flask was then charged with Pd(PPh₃)₄ (133 mg, 0.115 mmol) and degassed one more time by freeze-thaw-pump cycles. The reaction mixture was stirred at 60° C. for 2 h. The reaction mixture was diluted with CH₂Cl₂, washed with water (3×50 mL) and brine, dried (Na₂SO₄) and concentrated. Column chromatography (silica, 4:1 CH₂Cl₂/hexanes) provided a royal-blue film (35.1 mg, 16%). ¹HNMR (CDCl₃, 400 MHz): 7.61-7.56 (m, ²H), 6.92-6.87 (m, ²H), 6.49 (t, J=3.4 Hz, ²H), 1.22 (s, ¹⁸H). The dyes were characterized by ¹HNMR using a Bruker 500 MHz Avance III HD with Cryoplatform NMR and processed by MestReNova software, Mestrelab Research, Santiago de Compostela, Spain. HRMS (MALDI-TOF) spectra were obtained with an AxION TOF, PerkinElmer, Waltham, MA, USA. Absorbance and fluorescence spectra were collected in dichloromethane on a DU 800 Spectrometer, Beckman Coulter, Pasadena, USA, and a Horiba Quantamaster 400 fluorimeter, Kyoto, Japan.

### 1.3. Polymer Dots (Pdots): Synthesis and Characterization

NIR emitting dye-doped Pdots were synthesized as described in Example 1. Oxygen sensing Pdots were synthesized with the same method, replacing PFBT with PFO and hydroporphyrins with Tris(4,7-diphenyl-1,10-phenanthroline) ruthenium (II) dichloride. Tris(4,7-diphenyl-1,10-phenanthroline) ruthenium (II) dichloride was not soluble in pure THF but was soluble in a 10% ethanol/THF mixture. Additionally, despite the dye being partially water soluble, the dye molecules were encapsulated in the Pdots with high efficiency and the Pdots showed no measurable dye leakage in aqueous buffers. Characterization of the synthesized Pdots was performed as described in Example 1.

### 1.4. Cryo-TEM Analyses of Pdots

Pdot samples were concentrated using a 3 kDa MWCO Amicon spin filters. Three microliter of the sample was loaded on glow discharged holey carbon grids (Quantifoil, Q1.2/1.3R, 300 mesh) and excess liquid was blotted away. Grids were plunge-frozen in liquid ethane using a Leica EMGP2 and stored in liquid nitrogen until further use. Grids were loaded on a 300 keV Krios G3i cryo-TEM, ThermoFisher Scientific, Eindhoven, NL. Micrographs were collected using the standard EPU software along with K3 direct electron detector and a bio-quantum energy filter (Gatan, Pleasanton, CA, USA), with 20 eV slit: at a nominal magnification of 130,000× and pixel size of 0.6795 Å/pixel. Further image processing, dimension estimation and visualization was performed using ImageJ.

### 1.5. Conjugation of Secondary Antibodies to Pdots

See Example 1 above for the materials and methods.

### 1.6. Construction of FLAG-FLS2 Transgenic Plants

See Example 1 above for the materials and methods.

1.7. Fluorescence Imaging of Antibody-Conjugated Pdots when Targeting the FLS2 Receptor in Plant Cells

See Example 1 above for the materials and methods.

### 1.8. Oxygen Sensitive Pdots

Following purification, the oxygen sensing pdots were placed into a 3 mL quartz cuvette and capped with a rubber septum and the initial fluorescence spectrum at an excitation wavelength of 344 nm was collected. Then, the solution was saturated with N₂ or O₂ for 5 min and the fluorescence spectrum was collected. The cycle of N₂ then O₂ saturation was completed 5 times.

### Results and Discussion

### 2.1. Synthesis and Characterization of Near Infrared Emitting Pdots

As introduced in Example 1, a series of hydroporphyrin fluorescent dyes can be used as dopants to produce Pdots with tunable red-to-near infrared emission. Using chlorin and bacteriochlorin as a base, systematic structural modifications can be used to tune the Pdots' emission properties. As introduced above, the absorption and emission wavelengths can be tuned across a broad spectral range by either (a) substitution at 3,13-pyrrolic positions of the macrocyclic ring with conjugated, electron withdrawing, or electron donating substituents, (b) installation of an additional ring on the periphery of the macromolecule (exocyclic ring), or (c) assembling of the chlorins and bacteriochlorins into strongly conjugated arrays, i.e., arrays where two macrocycles are connected by a linker which provides strong π-conjugation between subunits. FIG. 10 shows the molecular structures of 4,8-Dibromobenzo[1,2-c;4,5-c′]bis[1,2,5]thiadiazole (BBTD) dyes, which are characterized with longer emission wavelengths than porphyrins. A similar nanopreciptation method was used to form BBTD-doped Pdots with an emission wavelength range between 780 and 1000 nm. The availability of a new class of BBTD dyes greatly expands the wavelength range of luminescent Pdots and enables cellular and tissue imaging in plants.

### 2.2. Cryo-TEM of Antibody Coated Pdots

Cryo-TEM measurements revealed that the Pdots are substantially spherical (FIG. 11A) with an average diameter of 21±5 nm (n=200). This is consistent with dynamic light scattering (DLS) measurements which as expected show a slightly higher hydrodynamic diameter. Zeta potential measurements of these Pdots show them to have a negative surface charge of −35 mV, which is attributed to the presence of negatively charged carboxyl groups on the Pdots' surface. The presence of negatively charged carboxyl groups enables the use of an EDC coupling reaction to functionalize the Pdots with goat anti-rabbit IgG secondary antibody (See, e.g., FIG. 8A). FIG. 11B shows a Cryo-TEM image of the antibody coated Pdots. Compared to the uncoated Pdots, there are noticeable dark spots on the particles, as well as a Y-shaped of an antibody molecule. Zeta potential measurements of antibody-coated Pdots show lower negative surface charge of about −15 mV due to antibody conjugation which decrease the number of negatively charged carboxyl groups on the Pdots' surface.

### 2.3. BBTD-Doped Pdots

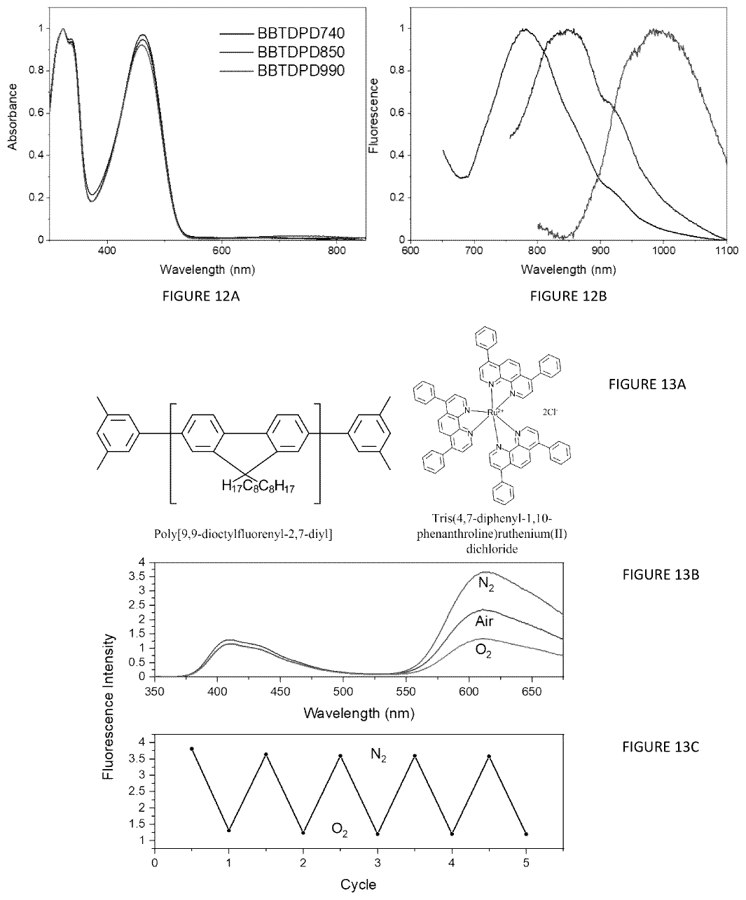

To overcome the limitations of fluorescent porphyrins, namely their inability to reach into the NIR-II window, an alternative series of benzo-bis-thiadiazole (BBTD) dyes were investigated. The absorbance of Pdots that are doped with BBTD dyes are shown in FIG. 12A. The spectra are dominated by the absorption peak of PFBT at 450 nm but the dyes also absorb between 620 nm to 700 nm. BBTDs utilize Donor-Acceptor-Donor (D-A-D) systems which possess an electron deficient central fluorophore covalently linked to two electron rich donors. This leads to large Stokes' shifts and long wavelength emission [Q. Yang, et al., 2017; Q. Yang, et al., 2018]. Increasing the donating strength of the donors enables tuning BBTD emission to longer wavelengths [Id.]. Since the base BBTD has a longer emission wavelength than chlorins or bacteriochlorins, the emission peak wavelength of BBTD-Pdots could be tuned from 700 nm all the way to 1000 nm while using a single excitation source at 450 nm (the peak excitation wavelength of PFBT). It should be noted that BBTD-Pdots show wider emission peaks than porphyrin-Pdots, which does limit their multiplexing potential. Additionally, their emission quantum yield is about 10-fold lower than the emission quantum yield of porphyrin-based Pdots, which range between 0.11 and 0.49. It is quite challenging to quantitatively measure the emission quantum yield of long wavelength emitting BBTD-Pdots due to the lack of suitable standards and the limited sensitivity of photodetectors in this wavelength range. The quantum yield of our BBTD-Pdots is found to be around 0.01 and additional improvements in their optical properties are still needed. Nevertheless, these drawbacks are outweighed by the long emission wavelengths of BBTD-Pdots which could provide superbly high signal-to-noise ratio in plant cell and tissue imaging applications.

### 2.4. Oxygen Sensitive Pdots

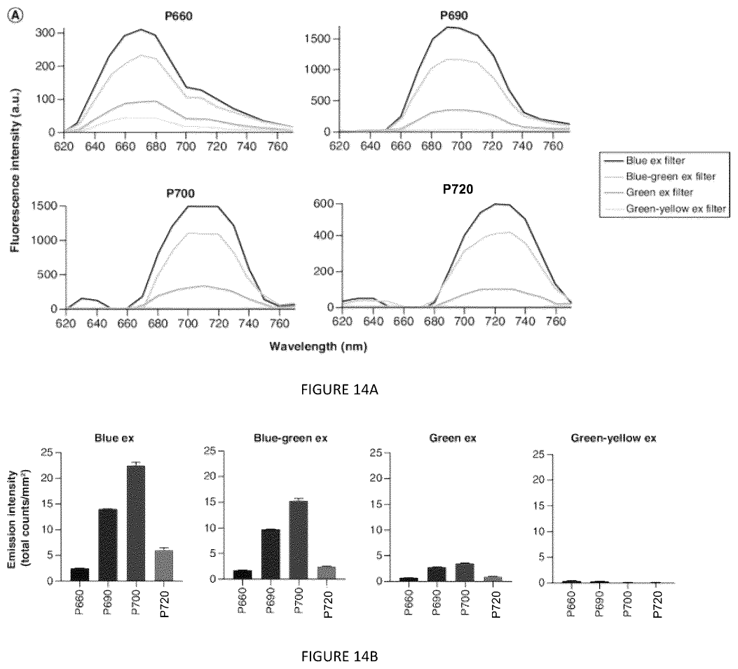

While studies to expand the utility of luminescent Pdots as imaging probes are on-going, there is a growing interest in the development of Pdot-based optochemical nanosensors. Oxygen-sensing Pdot based on tris (4,7-diphenyl-1,10-phenanthroline) ruthenium (II) dichloride Ru(dpp)₃²⁺ were synthesized. Ru(dpp)₃²⁺ features a highly efficient metal to ligand charge transfer (MLCT) transition which results in a long fluorescence lifetime, in the microsecond time scale, and high sensitivity to O₂ [H. J. Bolink, et al., 2006]. It exhibits six-times the quantum yield of Ru(bpy)₃²⁺ and is more resistant to water-induced substitution reactions [Id.]. In forming oxygen-sensitive Pdots, a blue-emitting Poly[9,9-dioctylfluorenyl-2,7-diyl] (PFO) polymer was used for better overlap with the absorption spectrum of Ru(dpp)₃²⁺. The structures of the polymer and dye are shown in FIG. 13A. Ru(dpp)₃²⁺ shows significant fluorescence enhancement when doped in the Pdot compared to the dye alone. The dye emission shows a strong response to N₂ and O₂ saturation while the polymer emission remains constant (FIG. 13B). To test the stability of Ru(dpp)₃²⁺-Pdots, an aqueous solution of Ru(dpp)₃²⁺-Pdots was subjected to five cycles of alternating O₂ and N₂ saturation. FIG. 13C shows that throughout the five cycles, fluorescence quenching by O₂ was completely reversible, showing that the Pdots are highly stable and effectively measure the level of oxygen under these conditions.

### Summary and Conclusions

This example expanded Pdots technology through the development and use of newly synthesized porphyrin and BBTD dyes. Conjugation of antibodies to Pdots is required for selective cellular imaging. Cryo-TEM measurements confirmed the presence of antibody molecules on the Pdots' surface. Furthermore, fluorescence imaging studies show that the antibody-conjugated Pdots successfully target FLAG-tagged FLS2 membrane receptors in genetically engineered leaf cells with minimal non-specific binding. Further, the ability to form Pdots with oxygen sensing capabilities was demonstrated.

### Example 3

### Experimental Section

### 1.1. Synthesis of Pdots

Deep-red and near-IR dyes used for doping Pdots, including phthalocyanine P700 or hydroporphyrin derivatives P660. P690 and P720, were synthesized as described in Example 1.

### 1.2. Animals

All in vivo procedures were conducted in compliance with the Guide for the Care and Use of Laboratory Animal Resources, US National Research Council, and approved by the local Animal Care and Use Committee (MIP-003-4-F). Female athymic nude mice (BALB/c-nu/nu mice) aged 6-10 weeks were purchased from Charles River Laboratories (MA, USA). During in vivo experiments, the mice were anesthetized with 2-3% isoflurane inhalation or 0.75 mg of sodium pentobarbital with intraperitoneal administration (Ovation Pharmaceuticals, Inc., IL, USA). Before in vivo fluorescence imaging, the mice were euthanized by carbon dioxide inhalation.

### 1.3. Fluorescence Imaging

In vitro, in vivo and ex vivo fluorescence images were obtained with a spectral imaging system (Maestro In-Vivo Imaging System, CRI, Inc., MA, USA). Four types of excitation band-pass filters (blue, 445-490 nm; blue-green, 480-520 nm; green, 503-555 nm; and green-yellow, 540-580 nm) and a long-pass orange filter over 610 nm (emission) were used. The built-in adjustable filter was automatically programmed to step from 620 nm to 770 nm in 10 nm increments, and images captured at each wavelength were taken at the same exposure time (in vitro, 500 ms; in vivo or ex vivo, 4000 ms). The acquired images were analyzed with built-in Maestro software, which separates Pdot signals from autofluorescence by a spectral unmixing algorithm using spectral libraries obtained from each of the injected Pdot solutions, and a composite image consisting of four Pdot signals and autofluorescence. For in vitro experiments, 100 μl of each Pdot (P660, P690, P700 and P720) or phosphate-buffered saline as an autofluorescence image was applied to a 96-well plate. For in vivo experiments, the mice were first anesthetized and then received an intracutaneous injection of 10 μl of each Pdot solution into one of four sites: the middle phalange of the left or right upper extremity and the left or right ear. Another mouse was given intracutaneous injections of each Pdot solution (P690 or P700), prepared as above, into one of two sites: the middle phalange of the left or right lower extremity. The location of the injection was changed for each mouse. After all drug injections were completed, the animals were euthanized, and the skin from the neck to the chest within the imaging area was removed. The mice were placed in the supine position, and the injection sites were covered with nonfluorescent black tape to avoid overwhelming the dynamic range of the camera with signals from the injection sites. The wavelength-resolved spectroscopic imaging was performed within 10 min after administration. After in vivo images were captured, lymph node resections were performed. All excised lymph nodes were evaluated by ex vivo spectrofluorometric imaging.

### Results and Discussion

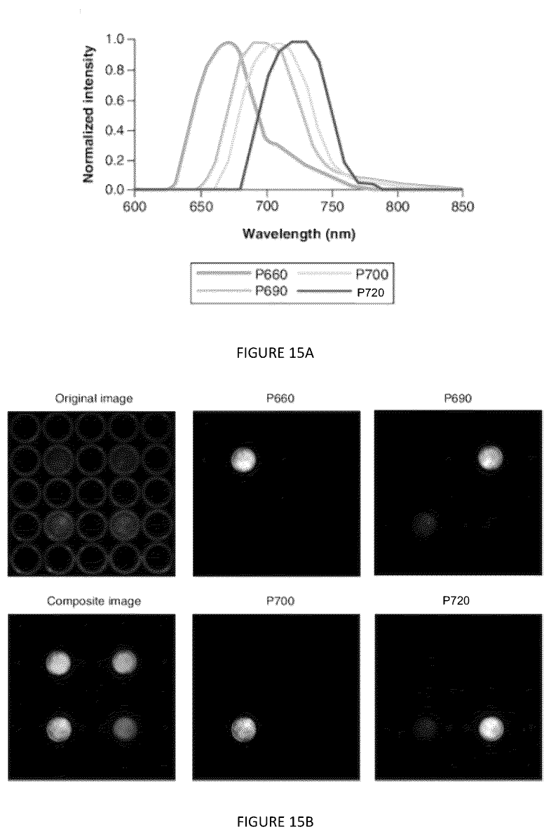

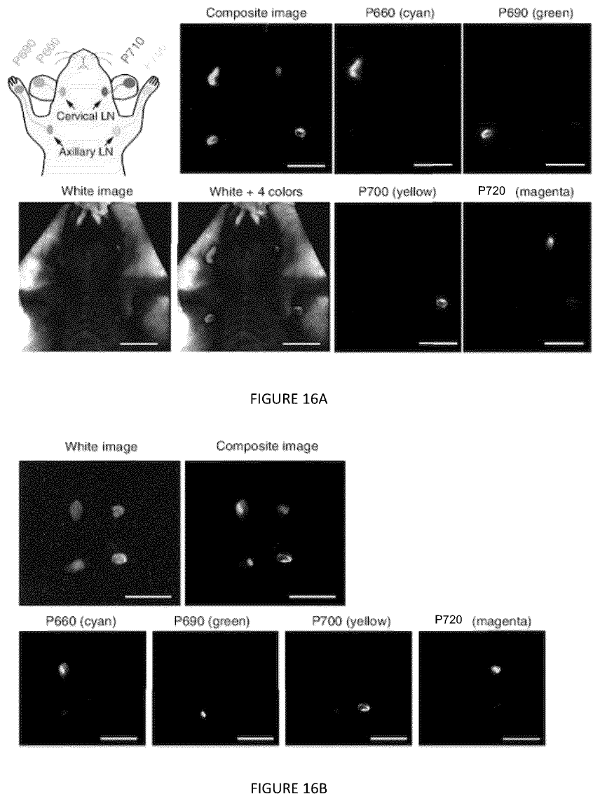

First, the emission intensity of each Pdot was measured and quantified using excitation band-pass filters (FIGS. 14A-14B). In all Pdots, the emission intensity was stronger for excitation light with shorter wavelengths in the visible light range (i.e., blue excitation band-pass filter, 445-490 nm, was the most efficient). Using the blue excitation filter with the exposure time held constant, P700 had the highest emission intensity and was about ten-times brighter than P660, which had the lowest emission intensity among the four Pdots. Next, it was determined whether four-color Pdots could be recognized with a single excitation light using a spectral imaging system. A library of four colors with different emission peaks was created using the spectra obtained from each Pdot solution (FIG. 15A). With a blue excitation band-pass filter of 445-490 nm, each of the four Pdots could be independently recognized and differentiated (FIG. 15B). Similarly, the four colors were successfully visualized separately using the blue-green (480-520 nm) or green (503-555 nm) excitation filter.

Next, fluorescence lymphangiography was performed in vivo using four Pdots in BALB/c-nu/nu mice. The mice were injected with P660 into the right ear and P720 into the left ear, respectively. Simultaneously, P690 and P700 were intracutaneously administered into the upper extremities. Four-color fluorescent lymphangiography using spectral fluorescence imaging successfully demonstrated visualization of lymphatic drainage to the primary lymph nodes (FIG. 16A). Ex vivo fluorescence imaging of resected regional lymph nodes demonstrated that all lymph nodes could be visualized (FIG. 16B). Furthermore, by rotating the Pdot administration, it was possible to distinguish the primary lymph nodes by fluorescence lymphangiography (Supplementary FIG. 3). Visualization of the popliteal lymph nodes, the primary lymph nodes of the lower legs, was also successfully obtained following Pdot injection into the middle phalange of both lower extremities (Supplementary FIG. 4). These results demonstrated that major primary lymph nodes (bilateral cervical and axillary lymph nodes) in the upper body of the mouse could be separately visualized in four color images using Pdots.

Accurate assessment of lymph node metastasis is important in the diagnosis and treatment of various cancers, including breast, gastric and cervical cancers and melanoma. For instance, the evaluation and treatment of sentinel nodes by a surgical resection is well established in breast cancer. However, an accurate understanding of the location of lymph nodes and lymphatic flow in the body is necessary to determine the optimal lymph node dissection, because these structures are part of a very complex and unpredictable system, varying greatly among patients. Lymphangiography utilizing Pdots could visualize these lymphatic networks and could be applied to identify sentinel node metastasis.

Successful simultaneous four-color lymphatic imaging was performed with the set of organic Pdots described herein. Although exposure time was long due to the need to cover a wide spectrum of emission, it was difficult to obtain multiple images from the same objects, probably due to the photobleaching. Additionally, Pdots wash out of lymph nodes relatively quickly, probably due to the hydrophilicity of the surface, yet these features tend to improve biocompatibility. Another potential limitation of Pdots is the need for short-wavelength emission light, which can be shorter than 500 nm. Such blue light barely penetrates into tissue and can be blocked by skin, making it difficult to see nodes through the skin. In this study, since neither of the Pdots could be excited with the blue excitation band-pass filter (445-490 nm) in BALB/c-nu/nu mice, the removal of skin was needed to obtain fluorescence images. This is not a serious limitation, as sentinel node imaging is often obtained intraoperatively with the overlying skin removed.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

H. J. Bolink, et al., Efficient and Stable Solid-State Light-Emitting Electrochemical Cell Using Tris(4,7-diphenyl-1,10-phenanthroline)ruthenium(II) Hexafluorophosphate, J. Am. Chem. Soc., 2006, 128, 46-47.
D. Chen, et al., Semiconducting Polymer Dots with Bright Narrow-Band Emission at 800 nm for Biological Applications, Chem. Sci., 2017, 8, 3390-3398, DOI: 10.1039/C7SC00441A.
L. Chen, et al., BODIPY-Based Donor/Donor-Acceptor System: Towards Highly Efficient Long-Wavelength-Excitable Near-IR Polymer Dots with Narrow and Strong Absorption Features, Angew. Chem., Int. Ed., 2019, 58, 7008-7012, DOI: 10.1002/anie.201902077.
S. Chen, et al., Simultaneous Near-Infrared and Green Fluorescence from Single Conjugated Polymer Dots with Aggregation-Induced Emission Fluorogen for Cell Imaging, J. Mater. Chem. B, 2018, 6, 7871-7876, DOI: 10.1039/C8TB02346H.
P. G. Frank, et al., Caveolin, Caveolae, and Endothelial Cell Function, Arteriosclerosis Thromb. Vasc. Biol., 2003, 23, 1161-1168, DOI: 10.1161/01.ATV.0000070546.16946.3A.
N. Gupta, et al., Near-Infrared-II Semiconducting Polymer Dots for Deep-Tissue Fluorescence Imaging, Wiley, 2021; Vol. 16, pp 175-184.
Y. Jin, et al., Near-Infrared Fluorescent Dye-Doped Semiconducting Polymer Dots, ACS Nano, 2011, 5, 1468-1475, DOI: 10.1021/nn103304m.
C.-S. Ke, et al., Molecular Engineering and Design of Semiconducting Polymer Dots with Narrow-Band, Near-Infrared Emission for in Vivo Biological Imaging, ACS Nano, 2017, 11, 3166-3177, DOI: 10.1021/acsnano.7b00215.
H. L. Kee, et al., Effects of Substituents on Synthetic Analogs of Chlorophylls. Part 1: Synthesis, Vibrational Properties and Excited-state Decay Characteristics, Photochem. Photobiol., 2007, 83, 1110-1124.
H. L. Kee, H. L., et al., Effects of Substituents on Synthetic Analogs of Chlorophylls. Part 2: Redox Properties, Optical Spectra and Electronic Structure, Photochem. Photobiol., 2007, 83, 1125-1143.
C. Kitamura, et al., Design of Narrow-Bandgap Polymers. Syntheses and Properties of Monomers and Polymers Containing Aromatic-Donor and o-Quinoid-Acceptor Units. Chem. of Mater., 1996, 8, 570-578.
M. Krayer, et al., Expanded Scope of Synthetic Bacteriochlorins via Improved Acid Catalysis Conditions and Diverse Dihydrodipyrrin-Acetals, J. Org. Chem., 2010, 75, 1016-1039, DOI: 10.1021/jo9025572,
J. K. Laha, et al., A New Route for Installing the Isocyclic Ring on Chlorins Yielding 13¹-Oxophorbines, J. Org. Chem., 2006, 71, 7049-7052.
H. H. Li, et al., Caveolae-Dependent and -Independent Uptake of Albumin in Cultured Rodent Pulmonary Endothelial Cells, PLoS One, 2013, 8, e81903 DOI: 10.1371/journal.pone.0081903.
Q. Li, et al., Novel small-molecule fluorophores for in vivo NIR-IIa and NIR-IIb imaging, Chem. Commun., 2020, 56, 3289-3292.
J. Lindsey, Near-infrared Tunable Bacteriochlorins Equipped for Bioorthogonal Labeling, New J. Chem., 2015, 39, 4534-4550, DOI: 10.1039/C5NJ00209E.
Z. Liu, et al., Narrow-band polymer dots with pronounced fluorescence fluctuations for dual-color super-resolution imagingstry, Nanoscale, 2020, 12, 7522-7526.
M. G. Murali, et al., New low band gap 2-(4-(trifluoromethyl)phenyl)-1H-benzo[d]imidazole and benzo[1,2-c;4,5-c′]bis[1,2,5]thiadiazole based conjugated polymers for organic photovoltaics. RSC Adv., 2014, 4, 44902-44910.
J. Pecher, et al., Nanoparticles of Conjugated Polymers, Chem. Rev., 2010, 110, 6260-6279, DOI: 10.1021/cr100132y.
N. R. Paisley, et al., Near-Infrared-Emitting Boron-Difluoride-Curcuminoid-Based Polymers Exhibiting Thermally Activated Delayed Fluorescence as Biological Imaging Probes, Angew. Chem., Int. Ed., 2021, 60, 18630-18638, DOI: 10.1002/anie.202103965.
C. Riahin, et al., Hydroporphyrin-Doped Near-Infrared-Emitting Polymer Dots for Cellular Fluorescence Imaging, ACS Appl. Mat. & Interfaces, 2022, 14, 18, 20790-20801.
C. V. Rohatgi, et al., Low-Bandgap Conjugated Polymer Dots for Near-Infrared Fluorescence Imaging, ACS Appl. Nano Mater., 2018, 1, 4801-4808, DOI: 10.1021/acsanm.8b01014.
H. Shi, et al., Ultrasmall Phosphorescent Polymer Dots for Ratiometric Oxygen Sensing and Photodynamic Cancer Therapy. Adv. Funct. Mater. 2014, 24, 4823-4830.
K. Sun, et al., Ultrabright Polymer-Dot Transducer Enabled Wireless Glucose Monitoring via a Smartphone. ACS Nano 2018, 12, 5176-5184.
M. Taniguchi, et al., Accessing the Near-Infrared Spectral Region with Stable, Synthetic, Wavelength-Tunable Bacteriochlorins, New J. Chem., 2008, 32, 947-958, DOI: 10.1039/b717803d.
M. Taniguchi, et al., Synthetic Chlorins, Possible Surrogates for Chlorophylls, Prepared by Derivatization of Porphyrins, Chem. Rev., 2017, 117, 344-535, DOI: 10.1021/acs.chemrev.5b00696.
D. Wang, et al., Bioconjugation of IgG Secondary Antibodies to Polymer Dots for Multicolor Subcellular Imaging, ACS Appl. Nano Mater., 2020, 3, 2214-2220.
E. Yang, et al., Photophysical Properties and Electronic Structure of Stable, Tunable Synthetic Bacteriochlorins: Extending the Features of Native Photosynthetic Pigments, J. Phys. Chem. B, 2011, 115, 10801-10806, DOI: 10.1021/jp205258s.
Q. Yang, et al., Rational Design of Molecular Fluorophores for Biological Imaging in the NIR-II Window, Adv. Mater., 2017, 29, 1605497.
Q. Yang, et al., Donor Engineering for NIR-II Molecular Fluorophores with Enhanced Fluorescent Performance, J. Am. Chem. Soc., 2018, 140, 1715-1724.
T. Xu, et al., How to Design Low Bandgap Polymers for Highly Efficient Organic Solar Cells, Mater. Today, 2014, 17, 11-15, DOI: 10.1016/j.mattod.2013.12.005.
Z. Yu, et al., Strongly Conjugated Hydroporphyrin Dyads: Extensive Modification of Hydroporphyrins' Properties by Expanding the Conjugated System, J. Org. Chem., 2014, 79, 7910-7925.
Q. Zhao, et al., Fluorescent/phosphorescent dual-emissive conjugated polymer dots for hypoxia bioimaging. Chem. Sci. 2015, 3, 1825-1831.

1. A method of making near infrared (NIR)-emitting polymer dots (Pdots), said method comprising:
dissolving at least one semiconducting polymer, optionally at least one additional component, and at least one doping dye in at least one water-miscible organic solvent to form a reaction mixture; and
coprecipitating the at least one semiconducting polymer, optionally at least one additional component, and at least one doping dye to form NIR-emitting Pdots by injecting the reaction mixture into a volume of water under sonication.

2. The method of claim 1, further comprising removing the at least one organic solvent and filtering the NIR-emitting dots to remove any large aggregates.

3. The method of claim 1, wherein the at least one semiconducting polymer comprises at least one of poly [(9,9-dioctylfluorenyl-2,7-diyl)-alt-co-(1,4-benzo-(2,1′,3)-thiadiazole)] (PFBT), Poly[9,9-dioctylfluorenyl-2,7-diyl] (PFO), and Poly [2-(5-cyano-methylhexyloxy)-1-4-phenylene] (CNPPP).

4. The method of claim 1, comprising the at least one additional component, wherein the at least one additional component comprises an amphiphilic surfactant, an electrolyte, a lipid, and/or dextran.

5. The method of claim 4, wherein the at least one additional component comprises at least one of Poly(styrene/maleic anhydride (PSMA), a poly(styrene) co-polymer with carboxylated poly (ethylene glycol) (PS-PEG-COOH), maleic anhydride, 1,2-distearoyl-sn-glycero-3-phosphoethanolamine (DSPE), 1,2-distearoyl-sn-glycero-3-phosphoethanolamine-PEG-carboxylic acid (DSPE-PEG-COOH), and dioleoyl-3-trimethylammonium propane (DOTAP).

6. The method of claim 1, wherein the NIR-emitting Pdots are substantially spherical with an average diameter of about 30 to about 65 nm, as determined using TEM.

7. The method of claim 1, wherein the doping dye is substantially encapsulated or embedded in a polymer matrix of the NIR-emitting Pdots.

8. The method of claim 1, wherein the at least one doping dye comprises a hydroporphyrin dye having varying auxochromes or a benzo-bis-thiadiazole (BBTD) dye having varying functional groups.

9. The method of claim 8, wherein the at least one doping dye comprises a hydroporphyrin dye selected from the group consisting of: a phthalocyanine; a red-emitting chlorin; a near-IR-emitting bacteriochlorins; a multimer with conjugated or cross-conjugated linkers, wherein the multimer is selected from chlorin-chlorin, bacteriochlorin-bacteriochlorin and chlorin-bacteriochlorin; a non-conjugated multimer with linkers, wherein the multimer is selected from chlorin-chlorin, bacteriochlorin-bacteriochlorin and chlorin-bacteriochlorin; and any combination thereof.

10. The method of claim 8, wherein the NIR-emitting Pdots comprising hydroporphyrin doping dyes can be excited by a single excitation wavelength and show narrow emission wavelengths ranging from about 640 to about 820 nm depending on the doping dye in the NIR-emitting Pdots.

11. The method of claim 8, wherein the NIR-emitting Pdots comprising hydroporphyrin doping dyes have an emission quantum yield in a range from about 0.11 to about 0.49.

12. The method of claim 1, wherein the NIR-emitting Pdots have a surface that is functionalized with negatively charged carboxyl groups.

13. The method of claim 8, wherein the at least one doping dye comprises a benzo-bis-thiadiazole (BBTD) dye selected from the group consisting of 4,8-Dibromobenzo[1,2-c;4,5-c′]bis[1,2,5]thiadiazole (BBTD-Br₂), BBTD850, BBTD990, and BBTD780.

14. The method of claim 8, wherein the NIR-emitting Pdots comprising BBTD doping dyes can be excited by a single excitation wavelength and show narrow emission wavelengths ranging from about 700 to about 1000 nm depending on the doping dye in the NIR-emitting Pdots.

15. Near infrared (NIR)-emitting polymer dots (Pdots) comprising at least one semiconducting polymer, optionally at least one additional component, and at least one doping dye, wherein the at least one doping dye comprises a hydroporphyrin dye having varying auxochromes or a benzo-bis-thiadiazole (BBTD) dye having varying functional groups.

16. An antibody-Pdots conjugate comprising at least one NIR-emitting Pdot of claim 15 and at least one antibody.

17. A method of making an antibody-Pdots conjugate, said method comprising:
mixing an amount of a solution comprising NIR-emitting Pdots of claim 15 with a poly(ethylene) glycol (PEG) solution and a buffer to form a first mixture;
adding a solution comprising an antibody to the first mixture with mixing to form a second solution; and
adding an amount of N-(3-Dimethylaminopropyl)-NO-ethylcarbodiimide (EDC) with agitation to effectuate a coupling reaction to conjugate the NIR-emitting Pdots with the antibody.

18. The method of claim 17, wherein the NIR-emitting Pdots comprise at least one semiconducting polymer, at least one additional component, and at least one doping dye, wherein the at least one doping dye comprises a hydroporphyrin dye having varying auxochromes or a benzo-bis-thiadiazole (BBTD) dye having varying functional groups

19. A method of using NIR-emitting Pdots for enhanced fluorescence imaging of one or more targets, said method comprising incubating a mixture comprising one or more NIR-emitting Pdots of claim 15 for a time to effectuate incorporation of the one or more NIR-emitting Pdots into and/or onto the target, and imaging said target using fluorescence measurement means.

20. The method of claim 19, wherein the one or more targets comprise different receptors, cells, tumors, and lymph nodes.
